(19) United States
(12) Patent Application Publication
(10) Pub. No.: US 2008/0167387 A1
(43) Pub. Date: Jul. 10, 2008

(54) Microglia Facilitated Amyloidogenesis Assay

(75) Inventors: James Edward Finley, Quaker Hill, CT (US); Robert Brelsford Nelson, Gales Ferry, CT (US); Charles Edmond Nolan, Groton, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc.

(21) Appl. No.: 11/816,351

(22) PCT Filed: Feb. 22, 2006
(86) PCT No.: PCT/US2006/006203

§ 371 Date: Aug. 15, 2007

Related U.S. Application Data
(60) Provisional application No. 60/655,183, filed on Feb. 22, 2005.

(51) Int. Cl. A61K 45/00; C12Q 1/00; G01N 33/566

(52) U.S. Cl. 514/789; 435/4; 435/7.2

(57) ABSTRACT

The present invention describes methods to identify compounds that prevents or treat amyloid accumulation in the brain, as mediated by microglia or cells of macrophage lineage. The present invention further describes compositions containing such compounds, methods of preparing such compositions and methods of using such compositions. The compositions are useful for treating or preventing diseases caused by or associated with cell-mediated amyloid formation, and are particularly useful in treating or preventing neurodegenerative diseases, such as Alzheimer's Disease and bovine spongiform encephalopathy.

### FIELD OF THE INVENTION

The present invention relates to novel methods and assays for identifying pharmaceutically effective compounds that are useful in the treatment or prevention of neurodegenerative diseases including Alzheimer's disease, and prion-mediated diseases including human and bovine spongiform encephalopathy and Creutzfeld-Jacob disease. The methods and assays of the invention are particularly adaptable for high throughput screening. Pharmaceutical compounds identified according to the practice of the invention are useful to prevent or treat formation and accumulation of amyloid proteins in the brain.

The present invention further relates to pharmaceutical compositions containing such compounds, and to methods for preparing and administering such compositions.

### BACKGROUND OF THE INVENTION

Alzheimer's disease (AD) is a progressive, degenerative neurological disease, leading to severely impaired cognition. It is the most common cause of dementia in older persons in Western countries.

In its early stages, AD is manifested by mild memory loss and cognitive problems. As the disease progresses, cognition problems increase and begin to interfere with daily activities. Certain patients suffering from AD may also suffer from agnosia, anxiety, and frustration. In the middle stages of the disease, patients begin to lose their ability to work and require daily supervision. Many patients also develop language deficiency, loss of judgment, reason, and severe behavioral changes. As the disease continues to develop, patients suffering from AD often become rigid, mute, incontinent, bedridden and incapable of caring for themselves. A more detailed discussion of disease progression is provided in Harrison's Principles of Internal Medicine, 15^{th} edition, McGraw Hill, New York, 2001.

Because of its symptoms, AD also exacts a heavy emotional toll on patients' families and caretakers. At present, there are almost 3 to 4 million people suffering from AD in the United States alone. Additionally, as the number of people of age 65 and older continues to rise, the social and economic impacts of AD have also become very serious.

Prior to the onset of clinically observable manifestations or symptoms of AD, neuropathologic markers appear in the brain of persons at risk from AD, and such marker conditions can continue to progress for decades in the adult brain before clinical onset of symptoms. While not every person who exhibits early or even advanced marker “pathology” will eventually progress to clinically recognized AD, the presence of certain markers, particularly at a high level, predictably correlates with eventual onset of disease. (See, for example, H. Soares, workshop presentation, “Diagnostic Accuracy of Cerebrospinal Fluid A-beta 1-42, Total Tau and Phosphotau in Alzheimer's Disease”, Molecular Diagnostics, New Applications and Technologies Accelerating Drug Development, Feb. 7-8, 2005 at Princeton, N.J., concerning disease correlations with (1) elevated levels of phosphorylated tau and (2) decreased levels of circulating A-beta peptide 42-mer, as assayed from preserved samples of cerebrospinal fluid from patients enrolled in long period National Institutes of Health, Bethesda, Md., USA clinical evaluations).

An important pathologic marker which both precedes clinical AD and strongly correlates with onset of the recognizable disease state is the accumulation of cytoplasmic neurofibrillary tangles within brain neurons themselves. Such tangles, particularly prevalent in hippocampus and cortex, are composed of a protein known as tau, which often becomes phosphorylated, a further predictive marker of the disease (see Herrmann et al., European J. Neurology, v. 42, pp. 205-210, 1999). Tau protein is believed to participate in microtubule assembly in normal cells (Weingarten et al., Proc. Natl. Acad. Sci., v. 72, pp. 1858-1862, 1962). However, abnormal (or excessive) phosphorylation of tau protein leads to the assembly of neurofibrillary tangles with other cell components. Since cytoplasmic neurofibrillary tau tangles are formed within the neurons themselves, they are immensely disruptive of cellular function, and upon sufficient accumulation lead to cell disfunction and death (Terry et al., Ann. Neorol., v. 10, pp. 184-192, 1981). Not surprisingly, there is considerable evidence associating the progressive accumulation of tau tangles with AD and other neurodegenerative diseases, such as cerebral amyloid angiopathy (CAA) and prion-mediated diseases, including human and bovine spongiform encephalopathy (Sasaki et al., Am. J. Pathol. v. 153 pp 1149-1155, 1998, Ghetti et al., Proc Natl. Acad Sci, v. 93, pp. 744-748, 1996; Kunzi et al., J. Neuroscience, v. 22, pp. 7471-7477, 2002.

The other primary pathological marker which also both precedes clinical AD and strongly correlates with onset of the recognizable disease state is the accumulation of neuritic senile plaques. Senile plaques take the form of insoluble, extracellular aggregates made of protein and non-protein components. Although accumulation of senile plaques is extracellular to the neurons themselves, the aggregates eventually become very damaging to proper neuron function, leading eventually also to cell death. (Selkoe, D. J. Cold Spring Harbor Symposia on Quantitative Biology, v. 61, pp. 587-596, 1996). Therefore, prevention or elimination of the formation of senile plaques appears to be an essential step in both treating and preventing AD, and similar neurodegenerative diseases.

Although senile plaques may have different morphologies, they are predominantly composed of aggregated protein masses known as amyloid, which are defined by nonbranching, fibrillar proteins, arranged in a cross β-pleated sheet conformation. Additional components may be included, such as the proteins apolipoprotein E and proteoglycans, and/or non protein components such as alpha 1-antichymotrypsin and P-component.

The primary component of AD-associated amyloid is the A-beta peptide, a peptide that is typically 39-43 amino acid residues in length (although other specific lengths, including exopeptidase-modified variants, are well recognized in the art and the use thereof is also within the practice of the present invention), and which is generated from amyloid precursor protein (APP). APP protein is generally distributed in the intercellular spaces between brain neurons. APP and APP-like proteins are found throughout the body and may function as regulatory and cell adhesion proteins, Herzog et al., Eur. J. Cell Biol., v. 83, pp. 613-614, 2004. The mature APP protein itself is typically 695, 750 or 751 amino acid residues in length (various splice variants and truncations occur) and is often mutated in patients from families showing a genetic predisposition to Alzheimer's disease (for example, at positions 671 and 717, see A. M. Goate, Cell Mol Life Sci, v. 54, pp. 897-901, 1998; Armstrong et al., Neurosci. Lett., v. 370, pp. 241-243, 2004; and Rosenberg et al., Acta Neuropathol., v. 100, pp. 145-152, 2000. Cleavage of the A-beta peptide from within the amino acid sequence of the APP polypeptide (with the A-beta peptide consisting of approximately residues 671-713 thereof) is probably most commonly accomplished by the proteases beta secretase and gamma secretase, acting outside the neurons, and the inhibition of these enzymes may also provide a therapeutic approach to the prevention and treatment of Alzheimer's disease and other neurodegenerative diseases (the beta secretase cleavage site is KM-DA between 671M and 672D). Because of slight variation in the specificity of these secretase enzymes, 39-residue, 40 residue, 41 residue, 42 residue, and 43 residue A-beta peptides may result. Action of secretases on APP may occur extracellularly, or intracellularly including within organelles.

The exact length of an A-beta peptide has been considered important to its participation in AD pathology, or to the exact timing of its participation in pathological events. Additionally, the 40-residue and 42-residue peptides may be the most common species present in plaques, and these species are present in brain fluid samples in familial AD, i.e. cases where members of a family are known to be predisposed to the disease. R. Vassar, Subcell. Biochem. v. 38, pp. 79-103, 2005. Additionally, in carefully preserved cerebrospinal fluid samples taken from patients as their disease states progressed (see H. Soares, supra), the concentration of 42-residue peptide—but in a non aggregated state—was shown to decrease as the disease progressed.

It is important to understand that A-beta peptide may accumulate extracellularly in the human brain for decades, in a diffuse, soluble, and non-amyloid state, without causing clinically measurable neurodegenerative symptoms. In fact, even the aggregation of A-beta peptide may not lead to clinical symptoms, as long as the peptides have not assumed the dense fibrillar array structure of amyloid plaques—in which the A-beta peptides adopt a beta sheet conformation. It is believed that the aggregation of diffuse or soluble A-beta into insoluble plaques directly correlates with severity of the disease state, and is likely necessary for clinically measurable symptoms. Although the A-beta peptide may become very insoluble in buffer solutions, in vitro, at moderate solution concentrations, it may remain surprisingly stable and soluble, in a diffuse state, in brain tissue of otherwise healthy adults, accumulating for decades without harmful effect. Thus, although prevention of proteolytic formation of A-beta peptide (for example, by inhibition of beta and gamma secretase action on APP) is widely recognized as a promising approach to AD therapy, it remains critically important to understand the processes that trigger aggregation of already accumulated A-beta peptide, and to prevent its aggregation (as a mature pathologic fibrillar form) in the brain during adulthood. This is particularly so given that, for various reasons, therapeutic intervention in adult patients is likely only to be provided in cases where familial predisposition is suspected, or where several decades of adult life (and A-beta accumulation) have already transpired before preventative or remedial therapy is initiated.

A-beta deposition in the mammalian brain has been achieved in a number of transgenic mouse models, in which native or mutant forms of the amyloid precursor protein are overexpressed using neuronal promoters (Higgins, et al., Ann. N.Y. Acad. Sci., v. 695, pp. 224-227, 1993; Quon et al. Nature, v. 352, pp. 239-241, 1991). The progression and pathology of A-beta deposition occurring in several of these models mimics A-beta deposition in the human AD-afflicted brain in the several ways: (1) formation of diffuse A-beta deposits precedes formation of amyloid-containing plaque deposits; (2) amyloid-containing deposits tend to have a more restricted distribution in the brain than diffuse A-beta deposits, and occur most frequently in areas known to be important for learning and memory; and (3) amyloid-containing deposits are often associated with MHC class II-positive microglia and dystrophic neurites, whereas diffuse A-beta deposits are not. (Pazmany et al., Brain Res., v. 835, pp. 213-223, 1999; H. Nakanishi, M O I. Neuriobiol. v. 27, pp. 163-176, 2003; Perlmutter et al., J. Neurosci Res., v. 33, pp. 549-558, 1992).

One of the notable features seen in these animal models, and also as evidenced from human tissue pathology samples, is the presence of microglia cells on even the smallest amyloid-containing deposits. Microglia are immune system cells, of macrophage lineage, that are generally of diffuse distribution throughout the brain and serve the function of maintaining homeostatis within the central nervous system microenvironment. In particular, microglial cells are known to act as scavengers to remove debris after neuronal injury or cell death. Such cells make up a substantial portion of brain cell mass, and are often positive for MHC class II-glycoprotein. A conventional view in AD research has been that the aggregation of A-beta peptide from the soluble, diffuse state into the aggregated amyloid state may be a spontaneous event (Orpiszewski et al., J. Mol. Biol., v. 289, pp. 413-428, 1999; Chen et al. Front Biosci., v. 4, A9-A15, 1999), and that microglia cells are attracted to amyloid deposits after they have formed. The “spontaneous aggregation” theory is further based on the existence of in vitro models demonstrating that A-beta peptide mixed in solution and stirred under a wide range of appropriate conditions can adopt an amyloid configuration on its own. However, the self-aggregation model is difficult to reconcile with the observations that (1) amyloid formation is often highest in particular cortex and hippocampal regions strongly associated with cognitive function, but rarely occurs in others, and (2) because amyloid formation may be delayed for decades following the appearance of A-beta deposits in the brain, and (3) happens to greater or lesser degrees in particular individual adults.

The present invention is based, at least in part, on the discovery that microglial cells routinely contact and/or process diffuse A-beta peptide molecules in the adult brain, and that certain molecular biological pathways in microglial cells contribute to such interactions and processing. Although it is believed that one consequence of this action by microglial cells is to endocytose or phagocytose A-beta peptide from the intercellular spaces of brain tissue, an unfortunate consequence of this processing is that aggregation and assembly of A-beta peptide to amyloid can also be facilitated. Accordingly, prevention and treatment of numerous neurodegenerative diseases can be provided by modulating the action of microglial cells on amyloid and prion precursors.

### SUMMARY OF INVENTION

The present invention relates in part to the recognition that microglial cells routinely process diffuse deposits of A-beta peptide molecules, and other accumulated proteins and peptide molecules, in order to maintain a proper intercellular environment in the brain. Numerous intracellular and intercellular signaling pathways contribute to the action of microglial cells on A-beta peptide, and on other accumulated or denatured peptides and proteins. The present invention is directed to affecting these signaling pathways to prevent or delay the appearance of actual symptoms of Alzheimer's Disease, and also to treat the disease once medically recognizable symptoms occur, in order to reverse the course of disease or prevent increase in severity of symptoms.

One such signaling pathway involves the numerous biological mechanisms whereby external signaling molecules, upon binding to a surface receptor of a microglial cell, can direct the increase or decrease in the effective concentration of cyclic nucleotides, such as cyclic adenosine monophosphate (“cAMP”, an important intracellular signaling compound) within microglial cells.

Other signaling pathways, useful in the practice of the invention, affect the overall behavior and phenotype of microglial cells. As elaborated below, mature microglial cells eventually express an “amyloid processing phenotype” which is typically associated with increased expression of MHC-type II cell surface antigen. Modifying the phenotypic behavior of such cells is very useful in preventing generation of brain amyoid.

In a representative embodiment, the invention therefore provides a method of identifying a compound useful for suppressing amyloid formation mediated by a cell, comprising contacting the cell with a candidate compound, adding amyloidogenic peptides or proteins, and comparing the level of amyloid formation mediated by the cell in the presence and absence of the candidate compound. In preferred examples the target cell is a microglial cell, and the amyloidogenic peptides are A-beta peptides associated with Alzheimer's disease.

In the practice of the invention, the term “compound” used in reference to suppression of amyloid formation is equivalent to a “pharmaceutically or biologically active substance”, that is, the term includes low molecular weight (typically under 1000 Daltons) organic compounds; nucleotides and nucleosides, whether synthetic or natural, including oligonucleotides assembled therefrom and nucleic acid molecules generally; proteins such as, but not limited to, cytokines and hormones, including peptide fragments thereof; organic polymers; and antibodies, and fragments of antibodies or synthetic proteins or peptides that are modeled on antibody domains and/or have one or more antibody-like functions. Compounds useful in the practice of the invention therefore include:

(1) compounds that increase the level of intracellular cyclic adenosine monophosphate (cAMP), such as dibutyrl cAMP, and 8-bromo cAMP; or potentially cAMP itself;

(2) compounds that inhibit the hydrolytic action of intracellular cAMP-specific phosphodiesterases, including but not limited to the examples of phosphodiesterase species or groups such as PDE4, PDE7 and PDE10;

(3A) compounds that interact positively with (i.e. are agonists for) cell surface receptors on brain microglial cells, and wherein activation of the receptor by the natural ligand or the agonist compound results, directly or indirectly, in an elevation of intracellular cAMP. Examples include prostaglandin E2 (as natural ligand) acting at the E2 receptor, and includes agonists that bind at receptor subtypes EP1, EP2 and EP4 of E2, most preferably EP2 and EP4, an example of which is butaprost;

(3B) Still further additional examples in category (3) are compounds that act as agonists at the beta-2 (nor) adrenergic receptor; such as albuterol, terbutaline, metaproterenol, and norepinephrine (although not selective for beta 2); and

(4) Pharmaceutical substances that interact with receptors whose activation otherwise results, directly or indirectly, in a decrease in intracellular cAMP, and wherein such pharmaceutical substances inhibit (are antagonists of) such receptors. In this regard, it will also be appreciated by those skilled in the art that since the biological effects of cAMP and cGMP are often opposed, it may be possible to directly affect a cGMP receptor (or cGMP-acting phosphodiesterase, or cGMP-interacting protein), oppositely compared to an approach that would be taken with a cAMP receptor, and the like, and still achieve an equivalent result in the practice of the present invention.

Additional compound categories include:

(5) Compounds which may or may not operate through a cAMP-mediated mechanism, as described above, for example, compounds that interact positively with (i.e. are agonists for) nuclear receptors in brain microglial cells, including glucocorticoids, non-glucocorticoid compounds that act at the glucocorticoid receptor, and dissociated agonists of glucocorticoid receptors (“DAGRs”, see U.S. Pat. No. 6,506,766); and

(6) Additional classes of compounds that may or may not operate through a cAMP-mediated mechanism, as described above, including proteins or compounds that act at various cell surface receptors of microglial cells, include transforming growth factor-beta (TGF-beta) and members of the TGF-beta-cytokine superfamily, and also agonists of the interleukin-10 receptor, or other Th2-type cytokine receptors. An example useful according to the practice of the invention is interleukin-4 (IL-4).

It should be noted that “receptor” according to the practice of the present invention therefore includes not only cell surface macromolecules (typically proteins and glycoproteins), but also intracellular proteins such as enzymes, or enzymes imbedded in the cell membrane or the membrane of an organelle, which upon binding of the “compound” as herein defined, whether directly or indirectly, effect a signal that causes a change in the state of the microglial cell.

Accordingly, diseases that can be prevented or treated according to the practice of the present invention include Alzheimer's disease, chronic inflammatory diseases such as psoriasis, uveitis and chronic pain, familial Mediterranean fever, familial hibernian fever, long-term hemodialysis, hereditary nonneuropathic amyloidosis and other neurodegenerative diseases, which include familial amyloid polyneuropathies (FAP), cerebral amyloid angiopathy, and prion-mediated diseases. Prion-mediated diseases further include spongiform encephalopathies, Creutzfeld-Jacob Disease, Gerstmann-Straussler-Scheinker syndrome, Fatal familial Insomnia, Kuru and Alpers Syndrome. It should be emphasized that both prevention and treatment of the aforementioned diseases are specifically comtemplated according to the practice of the invention. Given that the timeline for amyloid deposition is often very long, and that improvement in neural function can provide benefit even if disease pathology is already present, it will be immediately apparent to those skilled in the art that prevention of amyoid-involved diseases can be effected, that is, to prevent appearance of clinically relevant symptoms in patients. Similarly, by affecting the phenotype of microglial cells in the brain, treatment of those already diagnosed with an amyloid-involved disease can be accomplished including not only prevention of further deterioration in a patient's profile but also improvement in symptoms toward a healthy adult neurological state.

A further aspect of the invention is therefore represented by assay methods designed to detect such useful compounds, including the method of identifying a compound that suppresses or inhibits cell-mediated amyloid formation, comprising the steps of:

(a) preparing at least one cell capable of mediating amyloid formation from amyloidogenic peptides,

(b) mixing the cell with an effective amount of a candidate compound and amyloidogenic peptides,

(c) incubating the mixture for a sufficient length of time to allow amyloid formation,

(d) measuring the level of amyloid formation, and

(e) comparing the level of amyloid formation to the level in the absence of a candidate compound.

The present invention also provides pharmaceutical compositions containing compounds that suppress or inhibit cell-mediated amyloid formation, particularly by modifying the actions of microglial cells, and methods of preparing same.

### BRIEF DESCRIPTION OF FIGURES AND DRAWINGS

FIGS. 1A and B illustrate amyloid formation mediated by rat primary microglia from soluble Aβ (1-42) peptides. Amyloid aggregates were stained with thioflavin S (bright areas denoted by arrows), rat primary microglia were outlined with the microglial marker OX-42, and cell nuclei were stained with DAPI (denoted by asterisks).

FIG. 2 illustrates amyloid formation from soluble Aβ (1-42) peptides mediated by microglia. (A and D) rat primary microglia, (B and E) rat primary neurons, and (C and F) in the absence of any cells. Amyloid aggregates were stained with thioflavin S fluorescence (bright areas in A, B, and C), and soluble Aβ (1-42) peptides were stained with antibody 4G8, specific to Aβ peptides (bright areas in D, E, and F).

FIG. 3 illustrates the timecourse of amyloid formation from soluble Aβ (1-42) peptides as mediated by rat primary microglia, following the addition of soluble Aβ (1-42) peptides to rat primary microglia. Amyloid aggregates were labeled by thioflavin S fluorescence (bright areas).

FIG. 4 is a bar graph illustrating amyloid formation mediated by rat primary microglia when different concentrations of soluble Aβ (1-42) peptides were added to rat primary microglia. Amyloid aggregates were labeled by thioflavin S fluorescence (green).

FIG. 5A,B illustrate amyloid formation from soluble Aβ (1-42) peptides (A) in the presence of the different types of cells of monocytoid lineage, and (B) in a rat organotypic hippocampal slice. Amyloid aggregates were labeled by thioflavin S fluorescence (bright areas).

FIG. 6 illustrates amyloid formation from soluble Aβ (1-42) peptides (A) in the presence of undifferentiated embryonic stem cells, vs. (B) in the presence of macrophage-differentiated embryonic stem cells. Amyloid aggregates were stained with thioflavin S fluorescence (bright areas), and embryonic stem cells are identified in the background by macrophage antibody Mac3 and the cell nucleus marker DAPI.

FIG. 7A,B illustrates, at the electron microscope level, in rat primary microglia (A) the occurrence of amyloid aggregates from added soluble Aβ (1-42) peptides in long cytoplasmic “tubes” that often terminate in clathrin-coated heads (arrows), and (B) extracellular (asterisks) and intracellular (arrows) amyloid aggregates from added soluble Aβ (1-42) peptides mediated by rat primary microglia. Amyloid aggregates are identified by their characteristic fibrillar appearance at the electron microscope level of resolution.

FIG. 8A is a bar graph illustrating dibutyryl-cAMP (db-cAMP) inhibition of amyloid formation from soluble Aβ (1-42) peptides, as mediated by rat primary microglia. The inhibitory effect of db-cAMP against amyloid formation by microglia is concentration-dependent. The extent of amyloid aggregation was quantified by thioflavin S fluorescence staining.

FIG. 8B is a bar graph illustrating inhibition by 8-bromo-cAMP of amyloid formation from soluble Aβ (1-42) peptides, as mediated by rat primary microglia and the inhibitory effect of 8-bromo-cAMP against amyloid formation mediated by microglia. The extent of amyloid aggregation was quantified by thioflavin S fluorescence staining.

FIG. 9A is a bar graph illustrating the relative inhibitory effect of the prostaglandin EP2 sub-type on amyloid formation from soluble Aβ (1-42) peptides, as mediated by rat primary microglia. The extent of amyloid aggregation was quantified by thioflavin S fluorescence staining.

FIG. 9B is a bar graph illustrating the relative degree to which prostaglandin subtype EP2 increases the level of intracellular cAMP in rat primary microglia.

FIG. 10 is a bar graph illustrating that butaprost, a selective agonist for EP2 prostaglandin receptor, and sulprostone, a selective agonist for the EP3 prostaglandin receptor, inhibit amyloid formation from soluble Aβ (1-42) peptides, as mediated by rat primary microglia. The extent of amyloid aggregation was quantified by thioflavin S fluorescence staining.

FIG. 11 is a bar graph illustrating that selective agonists for the prostaglandin E2 receptors inhibit amyloid formation from soluble Aβ (1-42) peptides as mediated by rat primary microglia. The extent of amyloid aggregation was quantified by thioflavin S fluorescence staining.

FIG. 12 is a bar graph illustrating that selective inhibitors of various phosphodiesterases inhibit amyloid formation from soluble Aβ (1-42) peptides, as mediated by rat primary microglia, and the inhibitory effect is concentration-dependent. The extent of amyloid aggregation was quantified by thioflavin S fluorescence staining.

### DETAILED DESCRIPTION OF THE INVENTION

Microglia and cells of macrophage lineage have been implicated in the pathogenesis of many diseases or conditions, especially many inflammatory diseases, including psoriasis, uveitis; type 1 diabetes, septic shock, pain, migraine, rheumatoid arthritis, osteoarthritis, inflammatory bowel disease, asthma, immune complex diseases, multiple sclerosis, ischemic brain edema, toxic shock syndrome, heart failure, ulcerative colitis, atherosclerosis, glomerulonephritis, Paget's disease and osteoporosis, inflammatory sequelae of viral infections, oxidant induced lung injury, eczema, acute allograft rejection, and infection caused by invasive microorganisms.

Microglial cells possess numerous receptors, typically cell surface proteins, capable of accepting natural ligands, or pharmaceutical compounds that bind at the ligand binding site of the receptor. Pharmaceutically active compounds that bind at the receptor may act as agonists (having a similar role as a natural ligand) or as antagonists (opposing the function of the natural ligand), and additional pharmaceutically active compounds and natural signaling molecules (whether small molecular weight compounds or other proteins) can also bind to other sites on a receptor, or an adjacent cell surface macromolecule to modify either the nature of, or the intensity of, resultant signals. Such binding sites that are not the ligand binding site, per se, are often termed allosteric sites. Since most cell surface receptors are transmembrane proteins, or are associated with transmembrane protein proteins including ion channels, binding of a ligand normally causes a change in the conformation or catalytic behavior of the transmembrane protein, particularly as to portions thereof extending into a cell's interior (the cytoplasm) thereby triggering a change in the intracellular environment. Such an event may be commencement of catalytic activity on one or more cytoplasmic proteins or the release of further signaling molecules. Examples of microglial cell surface receptors whose properties are useful according to the practice of the present invention are discussed below. One notable group of signaling pathways includes the numerous biological mechanisms whereby external signaling molecules, upon binding to a surface receptor of a microglial cell, can direct the increase or decrease in the effective concentration of cyclic nucleotides, such as cAMP within microglial cells. Other signaling pathways affect the overall behavior of microglial cells.

As aforementioned, the present invention relates to the recognition that microglial cells routinely process diffuse deposits of A-beta peptide molecules, and other accumulated proteins and peptide molecules, in order to maintain a proper intercellular environment in the brain. Numerous intracellular and intercellular signaling pathways contribute to the action of microglial cells on A-beta peptide, and on other accumulated or denatured peptides and proteins. The present invention is directed to affecting these signaling pathways in the brain to prevent the appearance of actual symptoms of Alzheimer's Disease, and also to treat the disease once medically recognizable symptoms occur, in order to reverse the course of disease or prevent increase in severity of symptoms. The present invention is therefore directed to altering the processes whereby microglial cells contribute to the pathology of Alzheimer's disease.

The A-beta peptide (originally derived from APP protein, see below) initially deposits in human brain as diffuse non-fibrillar material having little adverse effect on surrounding neuropils (brain cells), and little overt effect on cognition or behavior. After an average delay of two or more decades of usually gradual deposition, however, Alzheimer's disease manifests clinically, and is associated with an increasing number of microglia-laden amyloid-containing deposits that dramatically compromise the surrounding neuropil. In these “neuritic plaques”, neuronal cell processes coming into direct contact with the amyloid core become distended and show abnormal accumulation of both organelles and phosphorylated microfilaments. Preventing the refolding of A-beta peptide into an amyloid conformation recognizable by amyloid labeling agents is herewith disclosed to prevent neuritic processes surrounding amyloid deposits from adopting an abnormal phenotype that likely compromises neuronal function.

The culture model we have developed demonstrates that microglia cells in the brain convert soluble A-beta (or A-beta non-fibrillar aggregates) into an amyloid conformation, suggesting that amyloid plaque formation is an active, cell-mediated process. The molecular mechanisms underlying cell-mediated amyloidogenesis are unknown in the art, however we believe it is likely that the process involves an increased expression of cell surface molecules on the microglia that the A-beta peptide interacts with. Additionally, referring to the phenotype of the involved microglial cells, expression of many immune system macrophage markers is low or absent in young brain but increases in aged brain and is abundant in the brain of Alzheimer's disease patients. We further conclude that increased expression in the Alzheimer's brain of molecules associated with antigen presentation, e.g. MHC-class II glycoprotein, provides a “scaffolding site” to which amyloidogenic peptides bind and that these binding sites facilitate the undesired refolding of A-beta peptides into an amyloid conformation. Accordingly, a further aspect of the invention involves suppressing an inappropriate phenotype in patients' microglial cells.

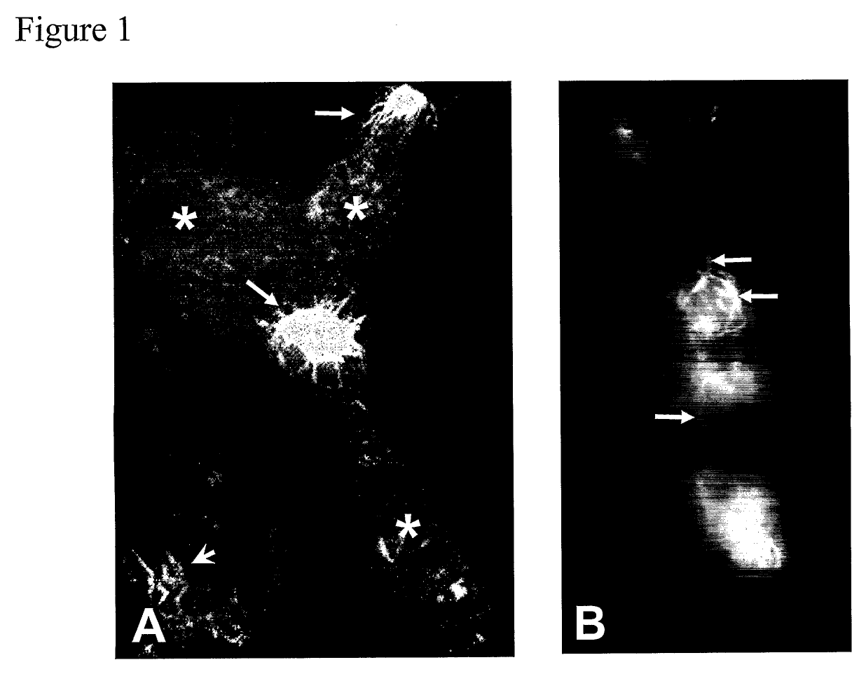

Also in regard of in vitro experiments, it is likely that the process of culturing microglia from brain removes the microglia from an environment in which immune-suppressing molecules maintain a low expression of immune system macrophage markers and phenotypic behaviors. For example, the complement system receptor CR3 is expressed by cultured microglia (visualized by the antibody OX-42 in FIG. 1), but constitutive expression of this marker on microglia in healthy brain is low. Further, a candidate molecule for maintaining suppression of immune system markers on microglia in normal brain is norepinephrine. Norepinephrine blocks cell-mediated amyloidogenesis, an effect we have traced to activation of the beta-2 adrenergic receptor using agonists specific for that receptor. Norepinephrine is known in the literature to promote a Th2 bias to the immune system, an effect also tied to agonist activity at the beta2 adrenergic receptor. Thus, in the context of Alzheimer's disease, loss of noradrenergic innervation of the brain through degeneration of the locus coeruleus (LC) is a hallmark lesion, and may in large part contribute to the increased expression of immune system markers in microglial, and otherwise in the Alzheimers affected brain. Supporting this conclusion, LC lesions in rat brain promote microglial activation. Accordingly, maintaining the brain microglial cells in an appropriate phenotype facilitates prevention of amyloidogenesis.

The cellular sites at which cell-mediated amyloidogenesis initiates are unknown, however electron micrographs suggest that clarthrin-associated portions of cell membrane are likely to be involved. This is based on the observation that amyloid containing “tubes” in the microglia frequently terminate in a clathrin-coated “head”. Since clathrin-coated pits are typically associated with cellular endocytic events, the profiles we have identified are suggestive of failed endocytosis, in which the formation of a clathrin-coated vesicle from a membrane was prevented by the seeding of amyloid formation at the clathrin “pit”, and that this event physically prevented “pinching off” of a althrin-coated vesicle from the membrane, resulting in the formation of a long membrane-surrounded amyloid-containing tube extending into the cell cytoplasm.

### Methods of Treating or Preventing Diseases or Conditions Caused by or Exhibiting Amyloid Formation

The present invention defines methods of treating or preventing diseases or conditions caused by or exhibiting amyloid formulation using a compound selected from the group consisting of (1) compounds that increase the level of intracellular cyclic adenosine monophosphate (cAMP), such as cAMP analogs, and certain inhibitors of cAMP-specific phosphodiesterases; and (2) compounds that interact with receptors whose activation or inactivation results in an elevation of intracellular of cAMP, such as agonists of prostaglandin receptors, and beta-2(nor)adrenergic receptors.

Amyloid formation has been implicated in various diseases and conditions, including, without limitation, chronic inflammatory disease, familial Mediterranean fever, familial hibernian fever, long-term hemodialysis, hereditary nonneuropathic amyloidosis and certain neurodegenerative diseases, which include AD, familial amyloid polyneuropathies (FAP), cerebral amyloid angiopathy, and prion-mediated diseases. Prion-mediated diseases further encompass spongiform encephalopathies, Creutzfeld-Jacob Disease, Gerstmann-Straussler-Scheinker syndrome, Fatal familial Insomnia, Kuru and Alpers Syndrome. In particular, prion-mediated diseases include bovine spongiform encephalopathy, which is also known as Mad Cow Disease. Thus, one embodiment of the present invention is a method of treating or preventing a disease or condition as described above in a subject in need thereof, comprising administering to the subject an effective amount of at least one compound that suppresses or inhibits amyloid formation mediated by microglia or cells of macrophage lineage, wherein the disease or condition exhibits or is caused by cell mediated amyloid formation. Amyloidogenic peptides refer to peptides that may aggregate to form amyloid, either spontaneously or by a cell-mediated process.

One preferred embodiment of the present invention is a method of treating or preventing a neurodegenerative disease in a mammal in need thereof, comprising administering to the mammal an effective amount of at least one compound that suppresses or inhibits cell-mediated amyloid formation. An even more preferred embodiment is a method of treating or preventing AD in a mammal, preferably a human, in need thereof, comprising administering to the mammal an effective amount of a compound that suppresses or inhibits amyloid formation. Another preferred embodiment is a method of treating or preventing a prion-mediated disease in a mammal in need thereof, comprising administering to the mammal an effective amount of at least one compound that suppresses or inhibits cell-mediated amyloid formation.

Methods of Identifying Compounds Affecting Cell-Mediated Amyloid Formation

The present invention further describes methods of identifying a compound that affects cell-mediated amyloid formation from amyloidogenic peptides. In particular, the present invention describes methods of identifying a compound that suppresses, inhibits or prevents cell-mediated amyloid formation, especially amyloid formation mediated by microglia or cells of macrophage lineage. These methods comprise the steps of contacting a cell capable of mediating amyloid formation from amyloidogenic peptides with a candidate compound, and comparing or measuring the level of amyloid formation in the presence and absence of the candidate compound.

More specifically, the present invention describes methods of identifying a compound that suppresses, inhibits or prevents cell-mediated amyloid formation, comprising obtaining at least one cell capable of mediating amyloid formation, mixing the cell with an effective amount of at least one candidate compound and amyloidogenic peptides, incubating the mixture, detecting amyloid formation, and comparing or measuring the level of amyloid formation in the presence and absence of the candidate compound.

The methods described herein may also be used to identify a compound that promotes or increases amyloid formation from amyloidogenic peptides. Thus, another embodiment of the present invention is a method of identifying a compound that promotes or increases cell-mediated amyloid formation from amyloidogenic peptides.

The methods described above can also be used to identify or screen for multiple compounds. Thus, in another embodiment of the present invention, multiple compounds are screened to identify at least one compound that affects cell-mediated amyloid formation. This method comprises selecting multiple candidate compounds, contacting each candidate compound with at least one cell that mediates amyloid formation, and measuring or comparing the level of amyloid formation mediated by such cell, in the presence and absence of each candidate compound.

The methods described in the present invention provide a convenient approach to compare multiple compounds for their abilities to affect cell-mediated amyloid formation. Accordingly, a preferred embodiment is a high throughput screening assay for identifying one or multiple compounds that suppress, inhibit or prevent cell-mediated amyloid formation, comprising contacting each candidate compound with cells that mediate amyloid formation, comparing each candidate compound's ability to affect cell-mediated amyloid formation, especially its ability to suppress, inhibit or prevent cell-mediated amyloid formation. An even more preferred embodiment is an automated high throughput screening assay.

As described above, amyloid mediating cells include a variety of cells that could affect amyloid formation in vivo or in vitro. Examples of these cells include, without limitation, microglia and cells of macrophage lineage, preferably from a mammalian brain or central nervous system. Cells of macrophage lineage further include, without limitation, macrophage-differentiated embryonic stem cells, microglia or macrophage cell lines, peritoneal macrophages, astrocytes, and monocytes. Some preferred cells are microglia and macrophage-differentiated embryonic stem cells.

For use in the assays of the invention, cells that mediate amyloid formation can be obtained commercially from sources such as American Tissue Culture Collection (herein “ATCC”). Alternatively, cells, such as microglia and peritoneal macrophage, may be isolated or obtained from their natural sources. See Whittemore et al., Int J. Dev. Neurosci. 11: 755-64 (1993); Kluve-Beckerman et al., Am. J. Pathol. 155: 123-133 (1999). For example, cells can be isolated from tissues or organs of an animal, preferably the brain and central nervous system, which may be preferably mammals or birds. Cells so obtained can be further expanded and harvested via in vitro tissue culture which is routinely practiced in the field. See Bernice M. Martin, Tissue Culture Techniques (Birkhauser Verlag AG, 1994).

Cells described herein can be isolated, or can be in vivo, e.g. existing in an animal. Isolated cells can be in pure form or be mixed with other components. One embodiment is a cell that is contained in an isolated tissue or organ, or fragments or portions of such tissue or organ, obtained from an animal, or preferably obtained from a mammalian brain.

In addition to cells, the present invention may also use a fragment or portion of tissues or organs to identify a compound that affects cell-mediated amyloid formation. Preferably, such fragments or portions of fragments or portions of tissues or organs are obtained from the brain or nervous system of a mammal as long as such tissues or organs have the ability to mediate amyloid formation. An example of such tissues or organs is, without limitation, a slice of brain from a mammal.

For use in the practice of the invention, preferred peptides are those derived from amyloid precursor protein (“APP”) known as “Aβ peptides”. See Small et al., Ann N Y Acad. Sci. 695:169-74 (1993). Aβ peptides can vary in the number of amino acids they contain as long as such variants do not affect the process of cell-mediated amyloid formation. Some of the more preferred embodiments are Aβ peptides having at least 17 and up to 43 amino acids. More preferred embodiments are Aβ peptides having 1-42, 1-40 or 1-17 amino acids. Other amyloidogenic peptides include, without limitation, serum amyloid A proteins (SAA), prion proteins, their derivatives, and other peptides having similar properties. See Prusiner Crit. Rev Biochem Mol. Biol. 26: 397-438 (1991); Kluve-Beckerman et al., Biochem Biophys Res Commun 181:1097-102 (1991).

In addition, one or more amino acids in an amyloidogenic peptide may be added, deleted or substituted by other amino acids, or by analogs or derivatives of such amino acids, as long as such addition, deletion or substitution does not interfere with the cell-mediated amyloid formation process. For Aβ peptides, one or more amino acids may be deleted or replaced by other amino acids, including non-naturally occurring amino acids, but preferably by amino acids with similar properties. Certain amino acids in the peptides may be labeled by covalently linking such amino acids with a labeling agent, including, without limitation, a fluorescent marker, a marker specific for antibody binding, a sequence having specific affinity, radioactive materials, nucleotides, oligonucleotides, lipids and other appropriate markers.

Amyloidogenic peptides can be obtained from a variety of known sources, including commercial sources, such as American Peptides of Sunnyvale, Calif. Amyloidogenic peptides can be chemically synthesized using methods routinely practiced in the field. See e.g. Stewart et al. Solid Phase Peptide Synthesis (Pierce Chemical Co. 1984). They can also be obtained by use of an expression system, such as a bacterial, yeast or mammalian expression system. See e.g. Sambrook et al. Molecular Cloning (Cold Spring Harbor Press 1989); see also Kluve-Beckerman et al., Am. J. Pathol. 155: 123-133 (1999). They can also be obtained from natural sources, by isolation and purification, using standard methods routinely employed in the field to obtain peptides. See e.g. Scopes et al. Protein Purification: Principles and Practice (1996). For example, amyloidogenic peptides may be produced by other cells, such as smooth muscle cells, preferably vascular smooth muscle cells. Accordingly, the amyloidogenic peptides described in the present invention may be obtained by carrying out the described assay in the presence of a second type of cell, such as smooth muscle cells, capable of producing and secreting amyloidogenic peptides. In a preferred embodiment, the smooth muscle cell is a vascular smooth muscle cell obtained or derived from the brain.

Amyloid may be detected by a variety of methods, including, without limitation, microscopy, labeling agents and other techniques well known to a person skilled in the art. A labeling agent for amyloid is one or more chemicals that mark or label amyloid. Preferably, such a labeling agent can be used to measure amyloid formation quantitatively. A labeling agent may include, but is not limited to, polyclonal or monoclonal antibodies against amyloidogenic peptides or their derivatives, labeled antibodies, thioflavin S and Congo red. Other labeling agents are also available for use in the present invention, including, without limitation, antibodies specific to amyloid aggregates. See Miller et al., Biochemistry 42: 11682-11692 (2003); O'Nuallain and Wetzel, PNAS 99:1485-1490 (2002). A preferred labeling agent for amyloid formation is thioflavin S. Other methods may also be used to detect or measure amyloid formation. These methods include, but are not limited to, X-ray diffraction and crystallization (as described in U.S. Pat. No. 6,600,017), and atomic force microscopy, photochemical crosslinking, electron microscopy, circular dichroism, mass spectrometry, quasi-elastic light scattering, MRI of plaques in vivo, and in vivo imaging of thioflavine dye multiphoton microscopy.

### EXAMPLES

### Example 1

### Preparation of Rat Primary Microglia

To obtain rat brain primary microglia, 1-3 day old or newly born Sprague-Dawley rats were used. These rats were decapitated with large scissors. The heads were stored in Dulbeccos phosphate buffered saline (DPBS) obtained from Sigma of St. Louis, Mo. (catalog no. D8537) and placed in 150 mm dishes. The heads were then cut open to expose the brains, which were removed with forceps, and stored in freshly prepared DPBS.

Meninges were removed from both hemispheres of the newly prepared rat brains. The cerebellum and brainstem were also removed. Once the brains were cleared of meninges, the remaining hemispheres were stored in DPBS, and placed in 100 mm dishes obtained from Corning, Inc. of Corning, N.Y. The remaining hemispheres were minced with a sterile scalpel blade or a sterile single edge razor. 1 ml of trypsin stock at 10 mg/ml, obtained from Sigma of St. Louis, Mo. (catalog no. T-7309), was then added to the minced brain samples. The mixture was incubated for 15 minutes in a 370 incubator. Upon incubation, 1 ml of DNAse at 1 mg/ml was added into the mixture, and the mixture was triturated for approximately 1 minute until the mixture was uniform and without chunks.

The mixture was transferred to a 50 ml tube, and Dulbeccos Modified Eagles Media (DMEM), obtained from Invitrogen of Carlsbad, Calif. (catalog no. 1195-065), with 10% fetal bovine serum, penicillin/streptomycin at 0.1% and L-glutamine at 200 mM was added to the mixture so that the total volume was 50 ml. The mixture was diluted with DMEM so that the final concentration of the mixture was approximately one rat brain per 50 ml of the mixture. The diluted mixture was placed in culture flasks, 50 ml per flask. The mixture was incubated at 37° C. with 95% of oxygen and 5% of carbon dioxide. After 24 hours, the media were replaced with 50 ml of fresh media, and non-adhering cells were discarded. The flasks were then placed in a 37° C. incubator, with 95% oxygen and 5% carbon dioxide. The media were replaced again after about 1 week of incubation.

After approximately 10 days, microglia were observed attached to the glial cell layer, and floating in the media. To harvest microglia, the cap and neck of the flasks were tightly wrapped with parafilm and the flasks were placed in a shaker incubator at 37° C. from 4 hrs to overnight. After incubation, the microglia were collected by centrifugation.

To harvest more microglia, fresh DMEM with 10% fetal bovine serum, 0.1% penicillin/streptomycin and L-glutamine at 200 mM was added to the flasks, and the flasks were again placed in a 37° C. incubator, with 95% oxygen and 5% carbon dioxide. Microglia were harvested as described above, weekly for up to 3 weeks.

### Example 2

### Preparation of Mouse Peritoneal Macrophage

To obtain mouse peritoneal macrophages, mice were injected intravenously with 1 ml of 6% casein, obtained from Sigma of St. Louis, Mo. (catalog no. C8654). Four days after injection, the mice were sacrificed via CO2 asphyxiation, and their stomachs washed with 70% ethanol. Incisions were made at the base of the abdomen and the skin was pulled away from the abdominal area. Each mouse was injected with 15 ml of sterile DPBS mixed with 1% FBS at the peritoneal cavity and the injected media was mixed gently. The fluid was then removed using a 10 ml syringe and stored on ice. The fluid samples were collected and centrifuged at 1000 rpm, for 5 minutes. The pellets were resuspended in Macrophage Serum Free Media (MSFM) obtained from Invitrogen of Carlsbad, Calif. and plated at a concentration of 200,000 cells per well in 96 well black/clear plates. Cells were incubated for 4-5 hours to allow the macrophage to attach to the plates. Medium was replaced from time to time for further incubation and harvest.

### Example 3

### Preparation of Human Monocytes

To prepare human monocytes, 100 ml of blood was collected from a donor using a syringe containing 1.5 ml of heparin (30 Units/ml). The blood was diluted with 20 ml of Macrophage Serum Free Media (MSFM) obtained from Invitrogen of Carlsbad, Calif. (catalog no. 12065) (with 0.1% penicillin/streptomycin). 30 ml of diluted blood was plated over 15 ml of Lymphocyte Separation Media (LSM) obtained from ICN Biomedical Inc. of Costa Mesa, Calif. After centrifugation for 30 min at 1,400 rpm at room temperature, the mononuclear layer was at the plasma ficoll/hypaque interface.

Most of the upper layer was removed by vacuum without disturbing the monolayer. The monolayer was then removed and placed into a 50 ml conical tube containing 15 ml of LSM. After all layers were removed, the final volume was adjusted to 50 ml. The tubes were centrifuged for 8 min at 1,400 rpm and the supernatant was removed. The cells were then suspended in MSFM and washed twice. The cell pellets were suspended in 10 ml of MSFM and cells were counted using a hemocytometer. Cells were then plated into 96 well black/clear plate, at a concentration of about 200,000 cells/well. After a 2 hr incubation, supernatant was removed and cells were washed again with 100 ul of MSFM to remove non-adherent cells.

### Example 4

### Preparation of Macrophage/Microglia Derived from Embryonic Stem Cells

To prepare embryonic stem (ES) cell derived macrophage/microglia, four murine ES cell lines were used, including (i) the murine DBA-252 ES cell line derived from the DBA/1 Lac J inbred mouse strain, prepared in accordance with the methods described in Roach et al., Exp. Cell Res. 221: 520-525 (1995), (ii) the murine DBA-PGES1-22F cell line having a targeted homozygous mutation deleting the PGES-1 gene in DBA-252 ES cells, in accordance with the method described in Trebino et al., PNAS 100: 9044-9049 (2003); (iii) the murine DBA-p38-C69 cell line having a targeted homozygous mutation deleting the p38 gene in DBA-252 ES cells, as described in Allen et al., J. Exp. Med. 191: 859-70 (2000); and (iv) the DBA-IL10-75P ES cell line having a homozygous mutation of the IL-10 gene which was replaced by a Beta-Lactamase gene, as described in Mortensen et al., Mol. Cell. Biol. 12: 2391-2395 (1992).

Cells were placed on a primary embryonic fibroblast (PEF) feeder layer treated with mitomyocin C. The cells were maintained in stem cell medium (SCML) which contained Knockout D-MEM obtained from Invitrogen Life Technologies, Inc. of Carlsbad, Calif. (catalog no. 10829-018), supplemented with 15% ES cell qualified fetal calf serum obtained from Invitrogen Life Technologies, Inc. (catalog no. 10439-024), 0.1 mM 2-mercaptoethanol, 0.2 mM L-glutamine, 0.1 mM MEM non-essential amino acids obtained from Invitrogen Life Technologies, Inc. (catalog no. 11140-050), 1000 u/ml recombinant murine leukemia inhibitory factor (LIF) obtained from Chemicon of Temecula, Calif. (catalog no. ESG-1107), and 50 μg/ml gentamycin.

To obtain the targeted ES cell lines, 1.5×10⁷ DBA-252 ES cells were suspended in 400 μl SCML and electroporation was performed using 25 μg linearized PGES Knock out targeting vector and a BTX Electro Cell Manipulator 600. Following electroporation, the cells were suspended in SCML and plated onto a PEF feeder layer treated with mitomyocin C. Twenty-four hours after electroporation, 175 μg/ml G418 obtained from Invitrogen Life Technologies, Inc. (catalog no. 10131-035) and 2 μM gancyclovir were added to the SCML. After 7-9 days, G418 resistant colonies were picked, plated into individual wells of a 24-well tissue culture dish, and expanded into clonal ES cell lines. Transformed ES cell selection lines that contained homologous recombination were identified by Southern analysis.

To develop macrophage/microglia, the ES cell lines were removed from PEF feeders and plated onto gelatin coated tissue culture dishes for two days in I-SCML that contained a base medium of Iscove's MDM, obtained from Invitrogen Life Technologies, Inc. (catalog no. 31980-030) supplemented with 15% ES cell qualified fetal calf serum obtained from Invitrogen Life Technologies, Inc. (catalog no. 10439-024), 0.1 mM 2-mercaptoethanol, 0.2 mM L-glutamine, 0.1 mM MEM non-essential amino acids obtained from Invitrogen Life Technologies, Inc. (catalog no. 11140-050), 1000 u/ml recombinant murine leukemia inhibitory factor obtained from Chemicon and 50 μg/ml gentamycin. The plated ES cells were grown in suspension for 6 days to form cell aggregates (known as embryoid bodies or EBs).

The EBs were dissociated and plated in tissue culture dishes in Mac I medium that contained the base medium Iscove's MDM as described above, supplemented with 10% FBS, 5% PFHM-II obtained from Invitrogen Life Technologies, Inc. (catalog no. 12040-093), 2 mM L-glutamine, 3 ng/ml M-CSF obtained from R&D Systems, Minneapolis, Minn. (catalog no. 416-ML-050), 1 ng/ml IL-3 obtained from R&D Systems (catalog no. 403-ML-010), and 50 μg/ml gentamycin. When the cell population became confluent, the macrophage precursors were harvested every other day from day 14 through day 30 as non-adherent clusters.

Non-adherent clusters of macrophage precursors were harvested from the media by centrifugation. Cell pellets were resuspended in Mac II media that contained the base medium Iscove's MDM, supplemented with 10% FBS, 5% PFHM-II obtained from Invitrogen Life Technologies, Inc. (catalog no. 12040-093), 2 mM L-glutamine, 3 ng/ml M-CSF, and 50 μg/ml gentamycin. Using an FACS assay, cells having 80% or greater Mac3⁺ and F4/80⁺ were harvested and plated onto tissue culture dishes or multi-well dishes in Mac II media and matured for 5-7 days for further analysis.

### Example 5

### Preparation of Rat Organotypic Hippocampal Slices

Ten to eleven day old male, newly born rats were used as tissue donors for hippocampal slices. Slices were cultured using interface methods described in Stoppini et al, J. Neurosci. Methods 37: 173-182 (1991). Slices were cut to about 400□ and incubated at room temperature, for 60 minutes or more, before mounting on 0.□□ cell culture inserts. Slices were then incubated in Gey's solution containing 0.5% glucose, 1% penicillin/streptomycin, and 1.5% Fungizone® obtained from GibcoBRL, Rockville, Md., at pH 7.2. Slices were positioned 6 per filter and 6 filters per culture plate. Culture dishes were filled with 1 cc of minimum essential medium (MEM) obtained from GibcoBRL of Rockville, Md. (catalog no. 12360), 50 cc of glutamine, 25 cc of horse sera obtained from GibcoBRL of Rockville, Md. (catalog no. 26050-088), 25 cc of Hank's BSS, 0.5% glucose, 1% penicillin/streptomycin and Fungizone® which was discontinued after one week. Support media was changed after 24 hours and twice per week thereafter. Experimental media is serum-free MEM with glutamine, Hanks' BSS, 0.5% glucose, and 1% pen/strep and propidium iodide at 2 □M. Experiments were performed on slices cultured a minimum of 2 weeks. All slices were examined for fluorescence pre-experimentally with an inverted Zeiss microscope and rhodamine filter. Wells containing fluorescent slices were discarded. All slices were cultured a minimum of 2 weeks prior to use. Images were captured using a digital camera and PC and stored for evaluation. Images were evaluated for mean fluorescence. Excitotoxins and antagonists were added to the experimental incubation media in low I volumes using Hank's BSS as vehicles.

### Example 6

### Microglial-Facilitated Amyloidogenesis Assay

The following assay, and other assays useful in the practice of the invention are useful to detect compounds that interfere with, or modify, the effects of microglia cells on the amyloidogenic process, irrespective of underlying mechanism. Accordingly, the practice of the invention is not limited as to theory in terms of the underlying mechanisms whereby microglial cells act on A-beta peptide, and useful compounds can be selected for the medical practice of the invention without limitation as to mechanism.

Primary rat brain microglia prepared as described in Example 1, were plated into Falcon black/clear 96 well plates at a concentration of 2×10⁵ cells per well. The microglia were allowed to attach to cell walls after incubation at 37° C. for several hours to overnight. Fresh DMEM, with 10% fetal bovine serum, 0.1% penicillin/streptomycin and L-glutamine at 200 mM was used to replace the old media, while cells were gently washed to remove debris and dead cells.

Cells were pre-treated with a candidate compound for 1 hour, at a concentration of 100 μM compound per well. (The pre-treatment step is optional.) After pre-treatment, the candidate compound and Aβ (1-42) peptides were added to the pre-treated cells. The final concentration of Aβ (1-42) peptides was 10 μM. As controls, one sample had only Aβ (1-42) peptides at a concentration of 10 μM, and another sample had only media. The final volume for each well was about 100 μl/well.

Aβ (1-42) peptides and Aβ (1-40) peptides, obtained from American Peptides of Sunnyvale, Calif., were stored at −20° C. Before use, Aβ (1-42) peptides were allowed to warm to room temperature for 15 minutes. To dissolve the Aβ (1-42) peptides, 200 μl of sterile water was added to the peptides. Each sample of peptides was vortexed to dissolve the peptides. Each sample was then diluted with 19 mls of MSFM. The final concentration of Aβ (1-42) peptides was 10 μM.

The mixture of rat brain primary microglia, Aβ (1-42) peptides, and a candidate compound was incubated for 24 hrs., at 37° C. After incubation, microglia were fixed with 100 μl of 95% ethanol and 5% acetic acid, which was directly poured into the media, and then incubated for 5 min. The media were poured out, and another 100 μl of fresh fixative having 95% ethanol and 5% acetic acid was added to the microglia for 10 min.

Fixed cells were stained with 100 μl thioflavin S solution, obtained from Sigma of St Louis, Mo. for approximately 3-5 min. Thioflavin S solution was 0.125 mg/ml in 40% ethanol, filtered through #1 Whatman filter paper before use.

Cells were washed twice, each time for 5 min, with 100 μl of 70% ethanol. After the washes, the cells were examined using an FITC filter under a fluorescent microscope. For each well, 3 images were collected and images were captured on a Zeiss Axiovert 100 M, with a 10× objective, using a digital CCD camera, and using MicroMax obtained from Roper Instruments of Tuscon, Ariz. Each treatment condition was run in triplicate, with 3 images collected per well to avoid areas with sparse cell growth and debris. Relative fluorescence intensity was measured.

In connection with the operation of the above assay (and all other assays as herein described), the reader will immediately note that most assays that involve detecting the result of binding of a ligand to a receptor may be run in numerous modes, depending on the characteristics of the involved components [for example taking into account (1) the lability or solubility of components, or (2) whether whole cells are used or only membrane fragments, or (3) whether and how physical supports for the assays, such as 96-well plates, can be operated, and operated in an automatic or high-throughput mode, all to most effectively detect both binding and binding mediated events. Accordingly, it may be more effective to run the assays of the invention not as direct binding assays, but as competition assays, for example, in which an inhibitor compound is first bound and then competed off the target by the natural ligand or agonist compound, for example. Representative modes in which assays can be run are described according to the following.

Additionally, although the present Example employs the well known response of thioflavin S fluorescence to fibrillar protein arrays, the unique physical character of an amyloid deposit can be detected and quantified via numerous other means including chemical techniques and physical techniques, such as X-ray diffraction and crystallization, and atomic force microscopy, photochemical crosslinking, electron microscopy, circular dichroism, mass spectrometry, quasi-elastic light scattering, MRI of plaques in vivo, and in vivo imaging of thioflavine dye multiphoton microscopy. Additionally, in addition to thioflavins, alternative dyes and other chromophores can be used such as (polarizing Congo Red, and others, as is well known in the art.

### Example 7

### Visualization of Microglia Mediated Amyloid Formation

Rat brain primary microglia were prepared in accordance with the protocol described in Example 1. Microglia so prepared were used to measure the effect on amyloid formation mediated by the microglia in accordance with the method described in Example 6.

The results are shown in FIG. 1. Panel A of FIG. 1 describes amyloid formation after 18 hours incubation of Aβ(1-42) peptides in the presence of microglia at a relatively low density. Cells were fixed with ethanol and stained with several markers. Panel A shows the presence of thioflavin S-positive amyloid deposits. These deposits formed predominantly within, or in close association with microglia (bright structures denoted by arrows), though extracellular thioflavin S-positive material was observed as well (bright material denoted by arrowhead). OX-42 is used as a background marker to outline the microglia. OX-42 is a microglia/macrophage marker that recognizes the CR3 antigen, and is obtained from Abcam Ltd of Cambridge, UK. The nuclei of microglia are identified with the nuclear stain DAPI (marked by asterisks).

Panel B of FIG. 1 displays a single microglial cell and the presence of wispy, elongated amyloid formation that was stained with thioflavin S (highlighted by arrows) in and around the microglial cell. Panel B of FIG. 1 shows the amyloid formation in and around the microglial cell, indicating the typical pattern of amyloid formation mediated by the microglial cell.

### Example 8

### Difference between Cell-Mediated Amyloid Formation and Spontaneous Amyloid Formation

In this Example, cell-mediated amyloid formation was compared with spontaneous amyloid formation. Conditions had been previously established, under which spontaneous conversion of amyloidogenic peptides into an amyloid conformation occurred in vitro. Rat primary microglia were prepared in accordance with the method described in Example 1. Microglia-mediated amyloid formation was measured using the method described in Example 6. In addition, amyloid formation was measured in the absence of any microglia or in the presence of neurons, cells of non-macrophage lineage. Aβ (1-42) peptides were used to measure amyloid formation.

Panels A-F show freshly solubilized Aβ(1-42) peptides, incubated in MSFM media for 18 hours in the presence or absence of cells. The samples were fixed and visualized with two markers. Thioflavin S was used to label Aβ(1-42) in an amyloid conformation (panels A-C), and antibody 4G8 was used to label precipitated Aβ(1-42) peptide that is in either an amyloid or non-amyloid conformation (panels D-F).

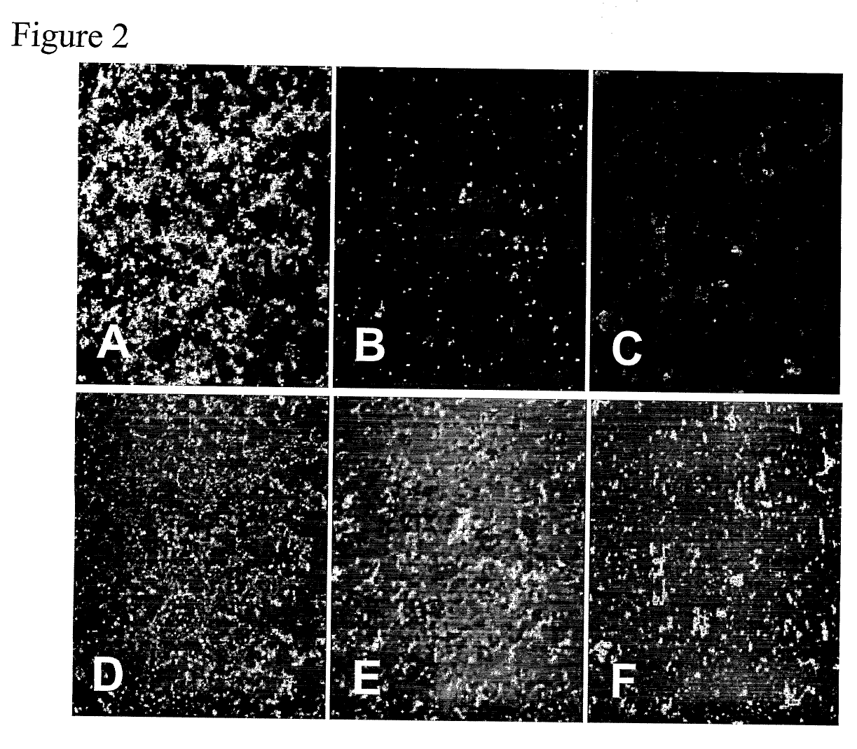

In FIG. 2, the bright areas in panel A demonstrate the formation of amyloid in the presence of microglia. The presence of bright areas in Panel D indicate that Aβ(1-42) peptide precipitated from solution and could subsequently be labelled with the 4G8 antibody. This precipitated Aβ(1-42) peptide was then converted extensively into amyloid (labelled by thioflavin S in panel A) in a process mediated by microglia. Cultured neurons rather than cultured microglia were incubated with soluble Aβ (1-42) peptides in FIG. 2, Panels B and E. Panel E shows that added Aβ (1-42) peptides also precipitate out of solution in the presence of cultured neurons. However, Panel B shows that much less Aβ(1-42) peptide is converted into amyloid in the presence of cultured neurons as assessed by lower thioflavin S labelling. FIG. 2 indicates that the microglia play an important role in mediating or facilitating amyloid formation, and that cell-mediated amyloid formation does not occur in a similar manner for all cells.

Another sample contained Aβ(1-42) peptides without any cells. The results are shown in Panels C and F of FIG. 2. The antibody 4G8 demonstrates the precipitation of Aβ(1-42) peptides out of solution in the absence of any cells (Panel F). After these Aβ(1-42) peptides at 10 μM precipitate from solution, they are deposited and fixed onto the plastic, and could be stained with antibody 4G8. However, as shown in Panel C of FIG. 2, the extent of spontaneous amyloid formation in the absence of any cells was minimal, compared to amyloid formation in the presence of microglia under the same conditions (Panel A).

### Example 9

### Timepoints for Microglia-Mediated Amyloid Formation

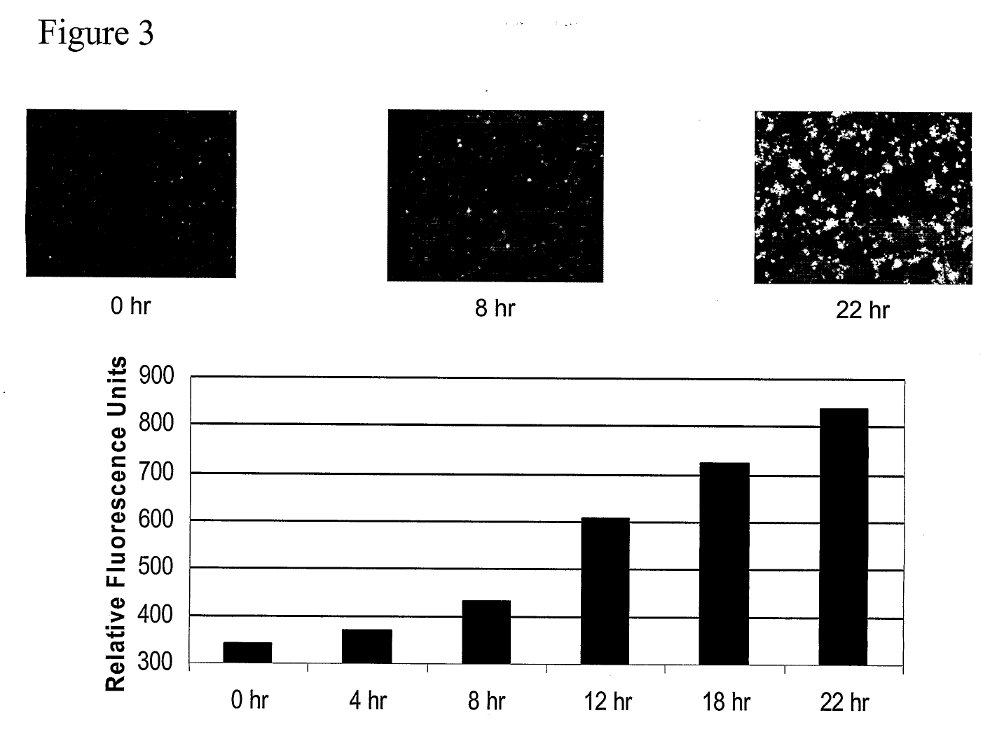

To examine the time needed for amyloid formation, 10 □M concentrations of Aβ(1-42) peptides and rat primary microglia were used. Rat primary microglia were obtained in accordance with the methods described in Example 1. The assay conditions were as described in Example 6. For each time point, a sample was prepared containing microglia mixed with 10 □M of Aβ(1-42) peptides, and incubated at 37° C. The samples were collected at different time points, after incubation of 4, 8 to a maximum of 22 hours. As shown in FIG. 3, in the presence of 10 □M of Aβ(1-42) peptides, amyloid formation mediated by microglia, as shown by thioflavin S staining, began to appear, developed more rapidly after 8 hours of incubation, and reached a maximal level after 22 hours of incubation.

### Example 10

### Effect of Aβ(1-42) Peptide Concentration on Amyloid Formation Mediated by Microglia

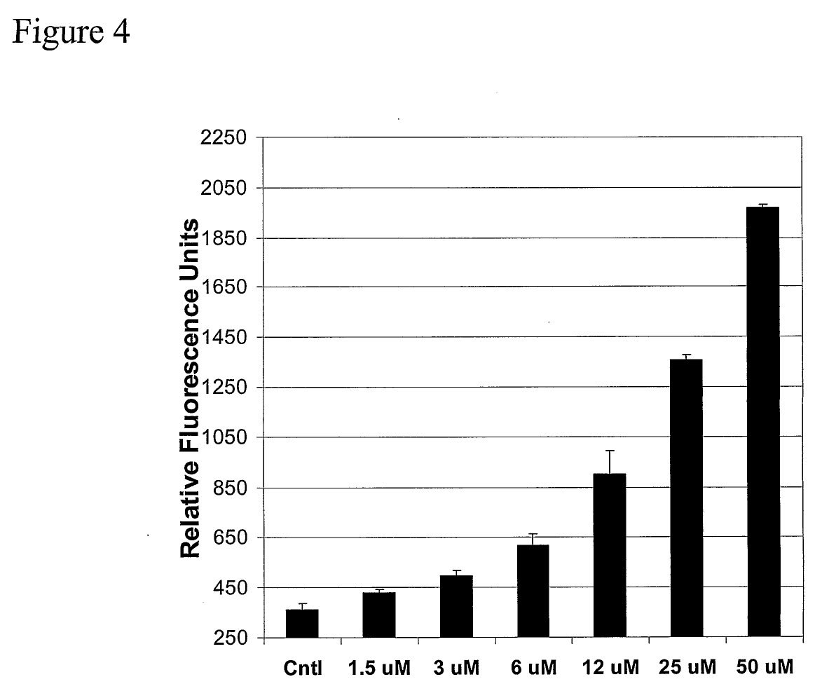

Amyloid formation mediated by microglia was tested using different concentrations of Aβ(1-42) peptides. Rat primary microglia were prepared in accordance with the methods described in Example 1. The assay was performed in accordance with the methods described in Example 6, without the use of any candidate compound, and without the step of pre-treatment. Different concentrations of Aβ(1-42) peptides were added to microglia and incubated at 37° C., for 18 hours. Cells were fixed and stained with Thioflavin S. As shown in FIG. 4, the level of amyloid formation, as indicated by the thioflavin S fluorescent signal, increased with increasing concentrations of Aβ(1-42) peptides.

### Example 11

### Amyloid Formation Mediated by Different Cells of Macrophage Lineage

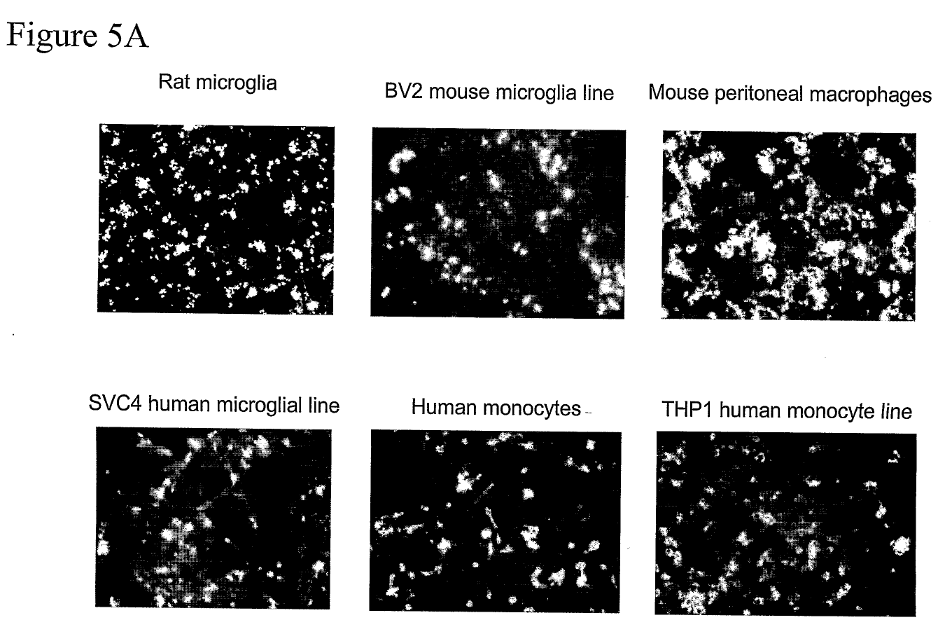

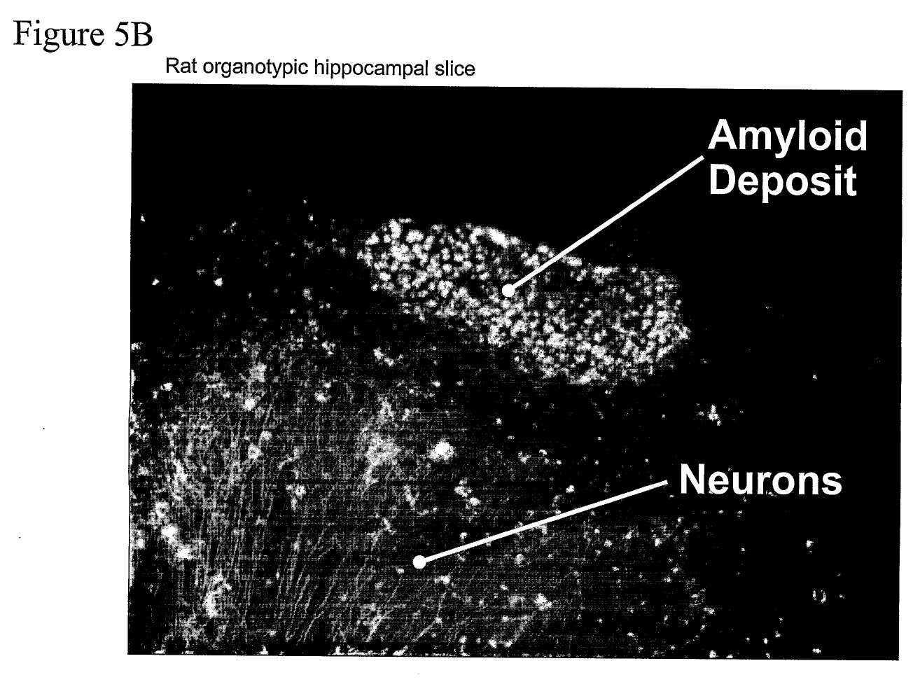

Amyloid formation was shown to be mediated by different cells of macrophage lineage. The cells tested included rat primary microglia prepared using the method described in Example 1, murine primary peritoneal macrophage prepared using the method described in Example 2, the human microglial cell line SVC4, human primary blood monocytes obtained in accordance with the method described in Example 3, murine microglial BV2 cell line obtained in accordance with the method described in Blasi et al., J. Neuroimmunol. 27:229-37 (1990), and macrophage-differentiated murine stem cells. The assay conditions were as described in Example 6, except that no candidate compound was used, and there was no pre-treatment of cells. The cells were mixed with Aβ(1-42) peptides, at a concentration of 10 μM and incubated at 37° C. for overnight. Thioflavin S staining (bright labelling) was used to show amyloid formation. As shown in FIG. 5A, each type of cell tested was able to mediate amyloid formation.

Cell-mediated amyloid formation was also measured using rat organotypic hippocampal slices. Rat organotypic hippocampal slices were prepared in accordance with the method described in Example 5. Cell-mediated amyloid formation was assayed in accordance with the method described in Example 6, without the use of a candidate compound. Rat organotypic hippocampal slices were mixed with 10 μM Aβ(1-42) peptides and incubated at 37° C. overnight. Amyloid formation was measured by thioflavin S staining. As shown in FIG. 5B, rat organotypic hippocampal slices facilitated amyloid formation, particularly in microglial-rich areas at the periphery of the slices.

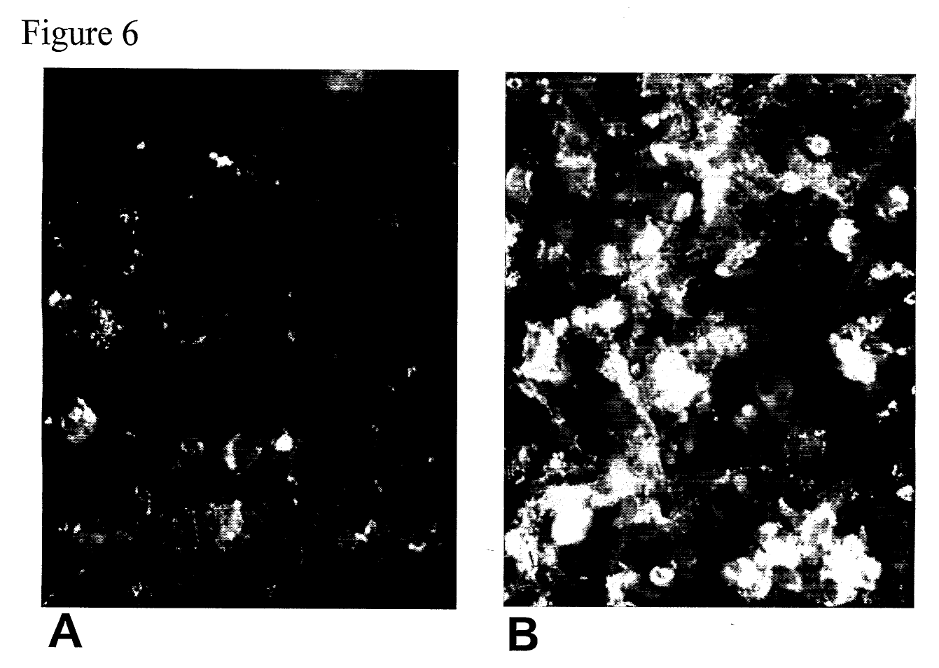

Cell-mediated amyloid formation was also measured using differentiated and undifferentiated embryonic stem cells. Embryonic stem cells (ES cells) were obtained as described above in Example 4. Cell-mediated amyloid formation was assayed in accordance with the method described in Example 6, without the use of a candidate compound, and without the pre-treatment step. Differentiated and undifferentiated ES cells were mixed with 10 μM Aβ(1-42) peptides. Each mixture was incubated at 37° C. overnight. Amyloid formation was measured by thioflavin S staining. As shown in FIG. 6, macrophage-differentiated ES cells facilitated amyloid formation. The level of amyloid formation mediated by undifferentiated ES cells was much lower.

### Example 12

### Properties of Cell-Mediated Amyloid Aggregates

Amyloid formation mediated by rat primary microglia was examined at the ultrastructural level. Rat primary microglia were prepared in accordance with the methods described in Example 1. Amyloid formation mediated by rat primary microglia was performed in accordance with the methods described in Example 6, without the use of a candidate compound and the step of pre-treatment with a candidate compound. The mixture of rat primary microglia and Aβ(1-42) peptides, at a concentration of 10 μM, was incubated at 37° C., for 18 hours. The sample was examined using transmission electron microscopy.

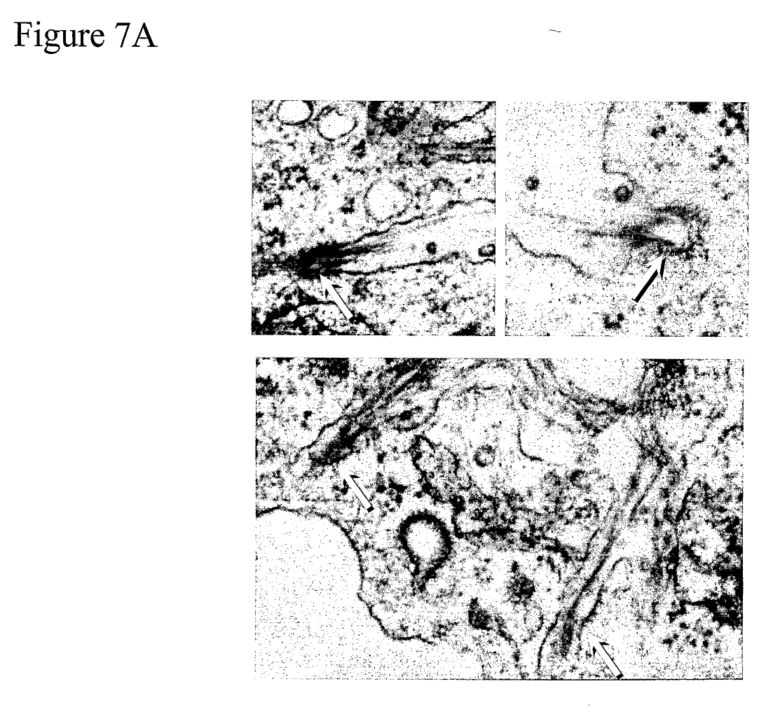

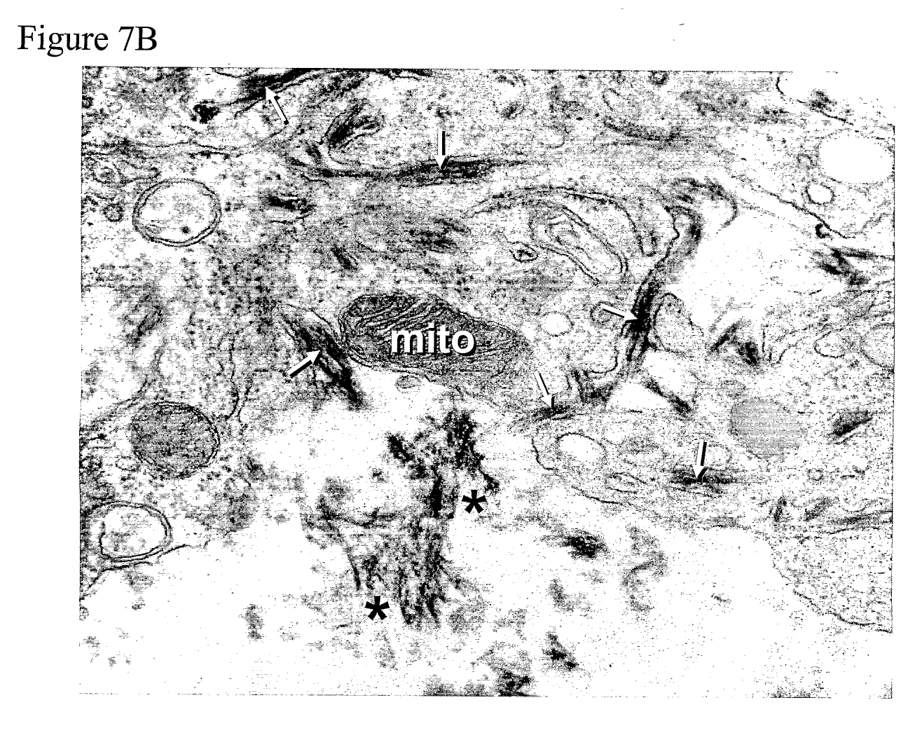

As shown in FIGS. 7A and 7B, in the presence of rat primary microglia, amyloid fibrils from Aβ(1-42) peptides were formed and observed in both extracellular (asterisks) and intracellular (arrows) sections. In addition, these electron micrographs revealed the presence of a network of amyloid-containing “tubes” throughout the microglial cytoplasm, reminiscent of the amyloid-containing membrane-encased structures present in neuritic plaques that form both in AD brains, and in the brains of transgenic animals overexpressing the amyloid precursor protein. The presence of fibrillar structures in the electron micrographs depicted in FIGS. 7A and 7B corroborate the presence of thioflavin S staining in light microscope pictures (e.g. FIG. 1) that fibrillar amyloid is being generated in the presence of microglia and macrophages.

### Example 13

### Inhibition of Microglia-Mediated Amyloid Formation by Enhancers of Intracellular cAMP

Using the method described in Example 6, the effect of cAMP and its analogs on cell-mediated amyloid formation was tested. Rat primary microglia were prepared using the method described in Example 1. One sample of rat primary microglia was treated with dibutyryl-cAMP (db-cAMP) at a concentration of 8-80 μM and another sample of rat primary microglia was treated with 8-bromo-cAMP (8-Br cAMP) at a concentration of 25-250 μM, starting one hour prior to addition of Aβ(1-42) peptides, at a concentration of 10 μM, As a positive control, a sample containing microglia without pretreatment was mixed with 10 μM Aβ(1-42) peptides, and as a baseline control, a second sample contained microglia without Aβ(1-42) peptides. Each mixture was incubated at 37° C., for 18 hours. Amyloid formation in each sample was measured by thioflavin S staining.

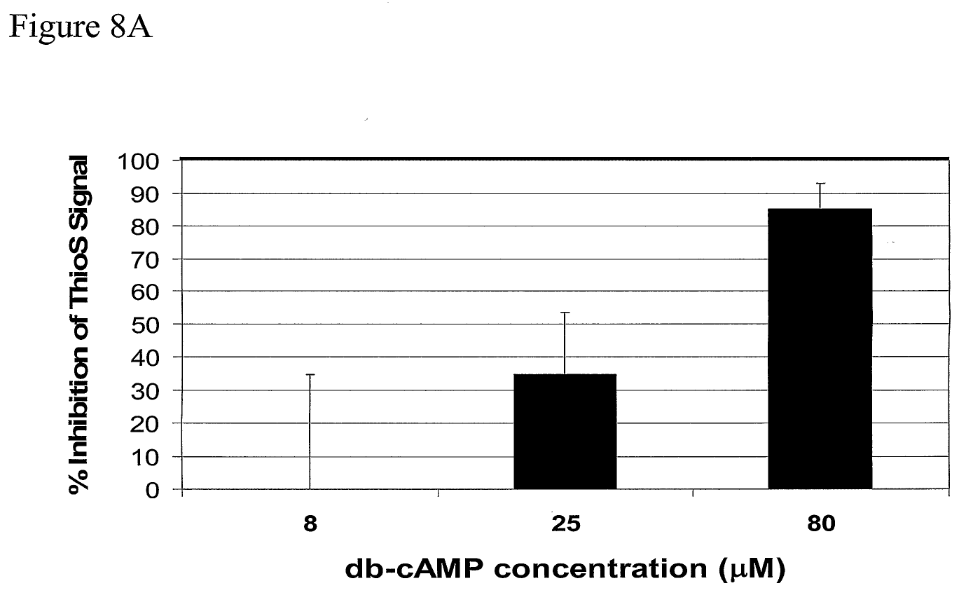

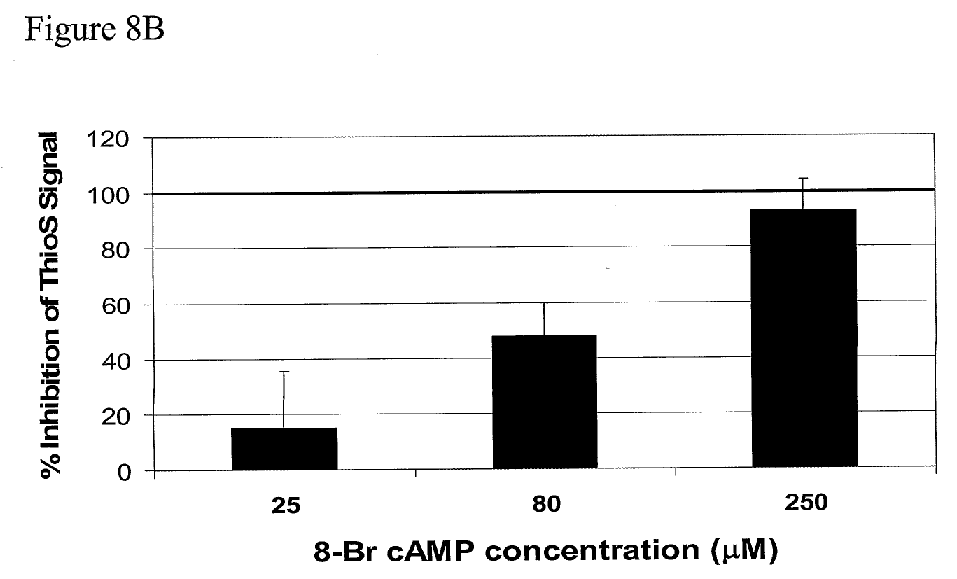

As shown in FIG. 8A, db-cAMP inhibited amyloid formation mediated by microglia. As shown in FIG. 8B, 8-Br cAMP inhibited amyloid formation mediated by microglia. The inhibitory effect of either db-cAMP or 8-Br cAMP against amyloid formation, mediated by microglia increased as the level of db-cAMP or 8-Br cAMP increased. The viability of microglia was also determined by measuring the level of the tetrazolium salt 3,[4,5-dimethylthiazol-2-yl]-2,5-diphenyltetrazolium bromide (MTT) reaction product, using the method provided by Trevigen Inc. of Gaithersburg, Md. The results showed that neither db-cAMP nor 8-Br cAMP had any effect on microglial viability.

### Example 14

### Inhibition of Cell-Mediated Amyloid Formation by Prostaglandins

Using the method described in Example 6, the effect of prostaglandins on cell-mediated amyloid formation was investigated. Rat primary microglia were prepared using the methods described in Example 1. Prostaglandin E2 (PGE2) was obtained from Calbiochem LaJolla, Calif. (catalog no. 538904) and tested for its ability to affect amyloid formation mediated by microglia, as measured by thioflavin S staining. The effect on amyloid formation was measured at two different concentrations, 0.32 μM and 3.2 μM.

To measure the effect on amyloid formation by PGE2, rat primary microglia were treated with the prostaglandins at a concentration of 0.32 μM or 3.2 μM, starting one hour prior to addition of Aβ(1-42) peptides at 10 μM. As a positive control, a sample containing microglia without pretreatment was mixed with 10 μM Aβ(1-42) peptides, and as a baseline control, a second sample contained microglia without Aβ(1-42) peptides. Each mixture was incubated at 37° C., for 18 hours. Amyloid formation in each sample was measured by thioflavin S staining.

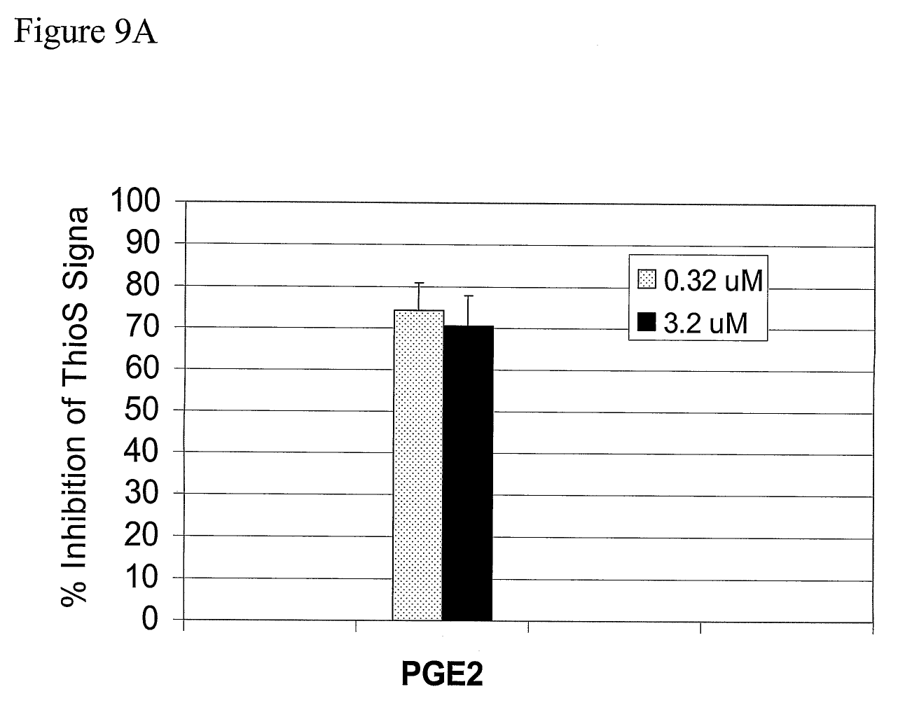

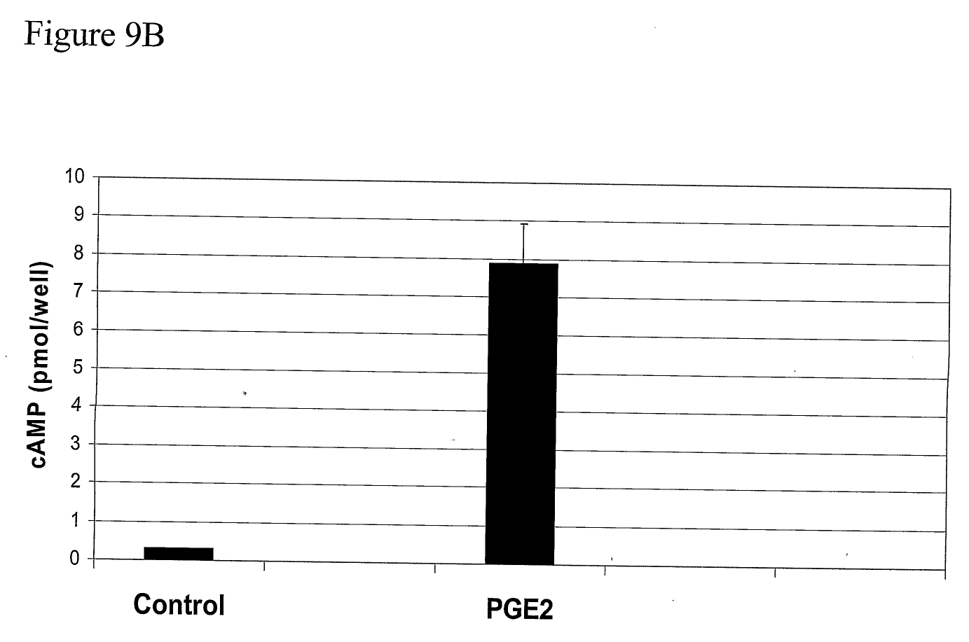

As shown in FIG. 9A, PGE2 was able to reduce amyloid formation mediated by rat primary macroglia.

The effect on the level of intracellular cAMP in microglia by PGE2 was also investigated. Rat primary microglia were prepared using the methods described in Example 1, and were exposed to PGE2, at a concentration of 1 μM, for about 20 minutes. As shown in FIG. 9B, PGE2 significantly increased the level of intracellular cAMP in microglia. Microglial viability was investigated by measuring the MTT reaction product in microglia. Exposure to PGE2 did not lead to any decrease in microglial viability.

### Example 15

### Inhibition of Cell-Mediated Amyloid Formation by Agonists of the Prostaglandin E2 Receptors

Using the method described in Example 6, the effect of agonists of the prostaglandin E2 receptors on cell-mediated amyloid formation was tested. Rat primary microglia were prepared as described in Example 1. One sample of rat primary microglia was treated with butaprost, a selective agonist at the prostaglandin E2 receptor EP2 subtype, at a concentration of 32 nM-10 μM, and another sample of rat primary microglia was treated with sulprostone, a selective agonist of the prostaglandin E2 receptor EP3 subtype, at a concentration of 32 nM-10 μM, one hour prior to addition of Aβ(1-42) peptides, at a concentration of 10 μM. As a positive control, a sample containing microglia without pretreatment was mixed with 10 μM Aβ(1-42) peptides, and as a baseline control, a second sample contained microglia without Aβ(1-42) peptides. Each mixture was by incubated at 37° C., for 18 hours. Amyloid formation in each sample was measured by thioflavin S staining.

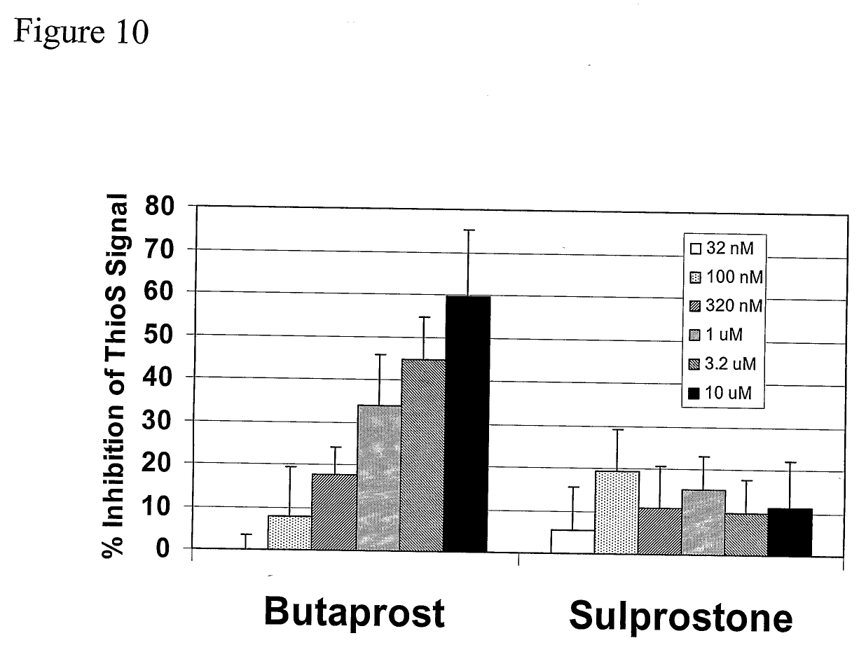

As shown in FIG. 10, butaprost inhibited amyloid formation mediated by microglia in a concentration-dependent manner. Sulprostone, in contrast had no clear effect on amyloid formation mediated by microglia. The viability of microglia was also determined by measuring the level of the tetrazolium salt 3, [4,5-dimethylthiazol-2-yl]-2,5-diphenyltetrazolium bromide (MTT) reaction product, using the method described by Trevigen Inc. of Gaithersburg, Md. The results showed that neither butaprost nor sulprostone had any effect on microglial viability.

Using the method described in Example 2, the effect of several additional selective agonists of the prostaglandin E2 receptors on cell-mediated amyloid formation was tested. Rat primary microglia were prepared using the method described in Example 1. Samples of rat primary microglia were treated with butaprost, Compound I or Compound II, selective agonists of the prostaglandin E2 receptors, at a concentration of 32 nM-10 μM, one hour prior to addition of Aβ(1-42) peptides, at a concentration of 10 μM. As a positive control, a sample containing microglia without pretreatment was mixed with 10 μM Aβ(1-42) peptides, and as a baseline control, a second sample contained microglia without Aβ(1-42) peptides. Each mixture was by incubated at 37° C., for 18 hours. Amyloid formation in each sample was measured by thioflavin S staining.

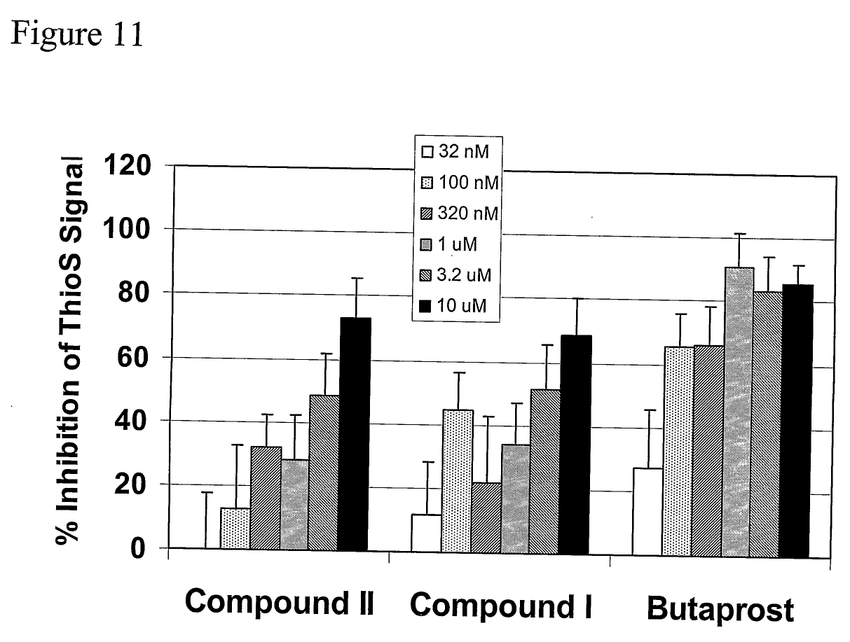

As shown in FIG. 11, treatment with butaprost, Compound I, or Compound II inhibited amyloid formation mediated by microglia in a concentration-dependent manner. The viability of microglia was also determined by measuring the level of the tetrazolium salt 3,[4,5-dimethylthiazol-2-yl]-2,5-diphenyltetrazolium bromide (MTT) reaction product, using the method described by Trevigen Inc. of Gaithersburg, Md. The results showed that neither butaprost nor sulprostone had any effect on microglial viability.

### Example 16

### Inhibition of Cell-Mediated Amyloid Formation by Inhibitors of Certain Phosphodiesterases

Using the methods described in Example 6, the effects of inhibitors of phosphodiesterases on cell-mediated amyloid formation was tested. Rat primary microglia were prepared as described in Example 1. Three different selective inhibitors of different phosphodiesterases (PDEs) were used, including papaverine, an inhibitor of PDE10, obtained from Sigma of St. Louis, Mo., Compound III (1-[4-(8′-chloro-2′,3′-dihydro-2′-oxospiro[cyclohexane-1,4′ (1′H)-quinazolin]-6′-yl)benzoyl]-4-methyl-peperazine, a selective inhibitor of PDE7 subtype a and subtype b, prepared in accordance with the methods described in the International Patent Application Publication Nos. WO02/076953 and WO021074754, and rolipram, a selective inhibitor of PDE4 subtype b and subtype d, obtained from Sigma Chemical Company.

Rat primary microglia were treated with the PDE inhibitors listed above, one hour prior to addition of Aβ(1-42) peptides, at a concentration of 10 μM. As a positive control, a sample containing microglia without pretreatment was mixed with 10 μM Aβ(1-42) peptides, and as a baseline control, a second sample contained microglia without Aβ(1-42) peptides. Each mixture was incubated at 37° C., for 18 hours. Amyloid formation in each sample was measured by thioflavin S staining.

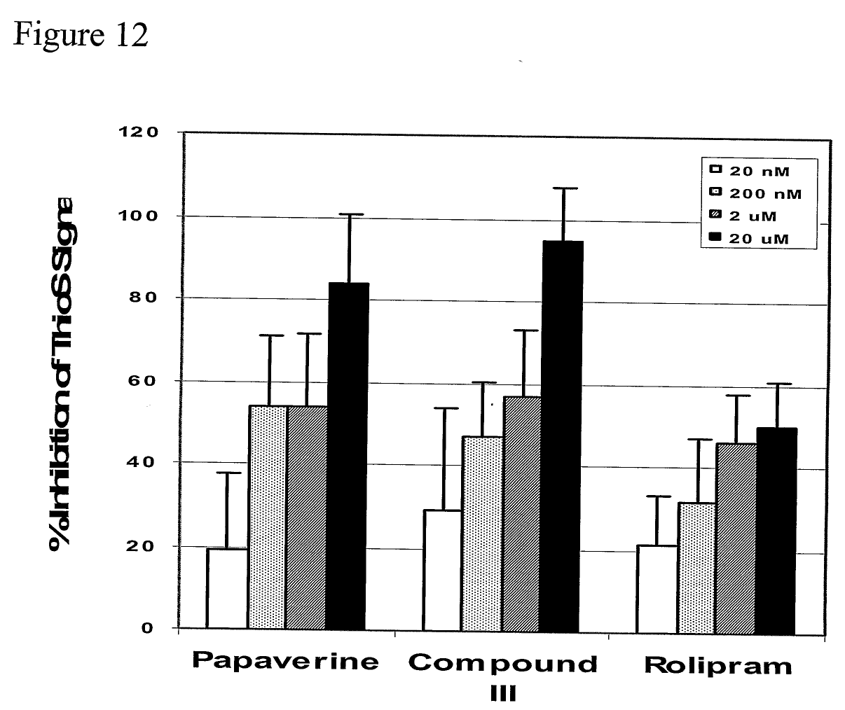

For each selective PDE inhibitor, the effect on amyloid formation mediated by microglia was measured in a concentration range of 20 nM-20 μM. As shown in FIG. 12, each of the selective PDE inhibitors inhibited amyloid formation mediated by microglia, as indicated by the decreased level of thioflavin S staining.

### Example 17

### Identification of an Inhibitor of Cell-Mediated Amyloid Formation

To identify a compound that inhibits cell-mediated amyloid formation, a candidate compound A having certain desired properties is selected. Primary rat microglia are prepared as described in Example 1. The assay is performed in accordance with the methods described in Example 6. A portion of primary rat microglia are pre-treated with 10 μM of compound A at 37° C., for one hour. After pre-treatment, the primary rat microglia are mixed with Aβ(1-42) peptides at a concentration of 10 μM, and Compound A at a concentration of 1 pM to 1M. The exact concentration of Compound A is subject to modification to achieve optimal effect. As a control, a portion of rat primary microglia without pretreatment are mixed with 10 μM, Aβ(1-42) peptides alone, while another sample contains only Aβ(1-42) peptides at a concentration of 10 μM, and Compound A at the corresponding concentration of 0.01 μM to 1M. Each mixture is incubated at 37° C. for 18 hours. Amyloid formation in each mixture is measured by thioflavin S staining. The effect of Compound A on microglial viability is also measured. Microglial viability is determined by measuring the MTT reaction product generated by microglia.

### Example 18

### Inhibition of Cell-Mediated Amyloid Formation by Selective Agonists of Prostaglandin E2 Receptor Subtype EP4

Using the methods described in Example 6, the effects of selective agonists of prostaglandin E2 receptor subtype EP4 on cell-mediated amyloid formation are tested. Rat primary microglia are prepared as described in Example 1. One or more selective agonists of the prostaglandin E2 receptor subtype EP4 are obtained using the methods described in U.S. Pat. Nos. 6,642,266, 6,610,719, and 6,552,067. Rat primary microglia are treated with the selective agonists of the prostaglandin E2 receptor subtype EP4, one hour prior to addition of Aβ(1-42) peptides, at a concentration of 10 μM. As a positive control, a sample containing microglia without pretreatment is mixed with 10 μM Aβ(1-42) peptides, and as a baseline control, a second sample contains microglia without Aβ(1-42) peptides. Each mixture is incubated at 37° C., for 18 hours. Amyloid formation in each sample is measured by thioflavin S staining.

### Example 19

### Effect of a Candidate Compound on Cell-Mediated Amyloid Formation

The following protocol is used to determine the effect of a candidate compound as identified using the methods described herein on cell-mediated amyloid formation in patients suffering AD.

A randomized, double-blind, placebo controlled study is conducted. Approximately 100 patients, both men and women, between the ages of 50 and 80, with a diagnosis of early or middle stage of AD, are recruited for participation in the study.

Patients are randomized for treatment with the candidate compound in the amount of 0.1 mg/day to 1000 mg/day, or a placebo for twelve weeks. Prior to randomization, patients are evaluated for dementia, using the evaluation guidelines provided the American Psychological Association's Ethical Principles of Psychologists and Code of Conduct (APA, 1992). The primary endpoint is a comparison between the treatment and placebo groups.

### Example 20

### Additional Assay Methodology and Options

The primary screening assays described herein are designed to detect “compounds”, as that term has been defined, that bind to cell surface receptors of microglial cells and affect the internal concentration of cAMP within said cells, or which cause other intracellular signaling effects. In binding assays, the effects of a particular compound are often compared to those of a natural ligand, and a test compound can be an agonist even if the effect produced is not as strong as that generated by the natural ligand. As described in detail below, such assays can be adapted to a high-throughput screening methodologies.

Binding assays may be performed either as direct binding assays or as competition binding assays. In a direct binding assay, a test compound is tested for binding either to the target receptor, or to a ligand of the target receptor. Competition binding assays, on the other hand, assess the ability of a test compound to compete with ligands or other test compounds for binding to the target receptor.

In a direct binding assay, a natural ligand or the receptor (or an enzyme) is contacted with a test compound under conditions that allow binding of the test compound to the ligand or the receptor. The binding may take place in solution or on a solid surface. Preferably, the test compound is previously labeled for detection. Any detectable group may be used for labeling, such as but not limited to, a luminescent, fluorescent, or radioactive isotope or group containing same, or a nonisotopic label, such as an enzyme or dye. After a period of incubation sufficient for binding to take place, the reaction is exposed to conditions and manipulations that remove excess or non-specifically bound test compound. Typically, this involves washing with an appropriate buffer. Finally, the presence of a ligand-test compound complex or a receptor-test compound complex is detected.

In a competition binding assay, test compounds are assayed for their ability to disrupt or enhance the binding of ligand to receptor. Labeled ligand may be mixed with cells expressing the receptors or membrane fragments thereof, for example, and placed under conditions in which the interaction between them would normally occur, either with or without the addition of the test compound. The amount of labeled ligand that binds receptor may be compared to the amount bound in the presence or absence of test compound.

An affinity binding assay may be performed using a microglial cell or membrane fragment which is immobilized to a solid support. Typically, the non-immobilized component of the binding reaction is labeled to enable detection. A variety of labeling methods are available and may be used, such as detection of luminescent, chromophoric, fluorescent, or radioactive isotopes or groups, or detection of nonisotopic labels, such as enzymes or dyes. In one preferred embodiment, the test compound is labeled with a fluorophore such as fluorescein isothiocyanate (FITC, available from Sigma Chemicals, St. Louis). The labeled test compounds, or ligand plus test compounds, are then allowed to contact with the solid support, under conditions that allow specific binding to occur. After the binding reaction has taken place, unbound and non-specifically bound test compounds are separated by means of washing the surface. Attachment of the binding partner to the solid phase can be accomplished in various ways known to those skilled in the art, including but not limited to chemical cross-linking, non-specific adhesion to a plastic surface, interaction with an antibody attached to the solid phase, interaction between a ligand attached to the binding partner (such as biotin) and a ligand-binding protein (such as avidin or streptavidin) attached to the solid phase, and the like.

Finally, the label remaining on the solid surface may be detected by any detection method known in the art. For example, if the test compound is labeled with a fluorophore, a fluorimeter may be used to detect complexes.

A labeled ligand may be mixed with cells that express a receptor, or less preferably, mixed with crude extracts obtained from such cells, and the test compound may be added. Isolated membranes may be used to identify compounds that interact with receptor. For example, in a typical experiment using isolated membranes, cells may be genetically engineered to overexpress a particular receptor. Membranes can be harvested by standard techniques and used in an in vitro binding assay. Labeled ligand (e.g., ¹²⁵I-labeled SLC) is bound to the membranes and assayed for specific activity; and specific binding is determined by comparison with binding assays performed in the presence of excess unlabeled (cold) ligand.

In another specific embodiment of this aspect of the invention, the solid support is membranes containing appropriate receptor attached to a microtiter dish. Test compounds, for example, cells that express library members are cultivated under conditions that allow expression of the library members in the microtiter dish. Library members that bind to the protein (or nucleic acid or derivative) are harvested. Such methods, are described by way of example in Parmley & Smith, 1988, Gene 73:305-318; Fowlkes et al., 1992, BioTechniques 13:422-427; PCT Publication No. WO 94/18318; and in other references cited herein.

Additionally, binding of ligand to receptor may be assayed in intact cells in animal models. A labeled ligand, for example, may be administered directly to an animal, with and without a test compound. The uptake of ligand may be measured in the presence and the absence of test compound. For these assays, host cells to which the test compound has been added may be genetically engineered to express the receptor and/or ligand, which may be transient, induced or constitutive, or stable. For the purposes of the screening methods of the present invention, a wide variety of host cells may be used including, but not limited to, tissue culture cells, mammalian cells, yeast cells, and bacteria. Thus, binding assays of the invention also include kinetic studies and measurements.

Chemotaxis assays may also be used as primary assays. One biological effect of the interaction between a microglial receptor and an attractant is the induction of the directional migration of cells expressing the receptor toward the particular attractant, a process known as chemotaxis. A chemotaxis assay, as described herein, may be used to screen compounds that interfere with the interaction of the receptor and the natural ligand/attractant. Such chemotaxis assays are adaptable to high throughput screening methods, and can thus be used in as a primary assay to identify useful compounds. A number of techniques have been developed to assay chemotactic migration (see, e.g., Leonard et al., 1995, “Measurement of a and β Chemokines”, in Current Protocols in Immunology, 6.12.1-6.12.28, Ed. Coligan et al., John Wiley & Sons, Inc. 1995).

The methods of the invention can routinely be performed in a high-throughput fashion for rapidly screening multiple test compounds. In particular, the cell systems used in such methods can be expressed and assayed in any multiple copy format known to those of skill in the art, including, but not limited to microtiter plates, spotting on agar plates, agar wells, spotting on chips and the like. Likewise, standard multiple manipulation techniques including but not limited to robotic handling techniques, can be utilized for multiple deposition of cells and/or test compounds.

After identification of a test compound that modulates the interaction of ligand with receptor, secondary screening assays may be used to further characterize the test compound for its effect on the biological activity. Various assays can be adapted to use as a secondary screen.

Diverse libraries of test compounds, including small molecule test compounds, may be utilized. For example, libraries may be commercially obtained from Specs and BioSpecs B. V. (Rijswijk, The Netherlands), Chembridge Corporation (San Diego, Calif.), Contract Service Company (Dolgoprudny, Moscow Region, Russia), Comgenex USA Inc. (Princeton, N.J.), Maybridge Chemicals Ltd. (Cornwall PL34 OHW, United Kingdom), and Asinex (Moscow, Russia).

Still further, combinatorial library methods known in the art, can be utilize, including, but not limited to: biological libraries; spatially addressable parallel solid phase or solution phase libraries; synthetic library methods requiring deconvolution; the “one-bead one-compound” library method; and synthetic library methods using affinity chromatography selection. An example of the biological library approach preferably involves a peptide library, while the other four approaches are applicable to peptide, non-peptide oligomer or small molecule libraries of compounds (Lam, 1997, Anticancer Drug Des. 12:145). Combinatorial libraries of test compounds, including small molecule test compounds, can be utilized, and may, for example, be generated as disclosed in Eichler & Houghten, 1995, Mol. Med. Today 1:174-180; Dolle, 1997, Mol. Divers. 2:223-236; and Lam, 1997, Anticancer Drug Des. 12:145-167.

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994. J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented from solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

Screening the libraries can be accomplished by any of a variety of commonly known methods. See, e.g., the following references, which disclose screening of peptide libraries: Parmley & Smith, 1989, Adv. Exp. Med. Biol. 251:215-218; Scott & Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al., Rebar & Pabo, 1993, Science 263:671-673; and PCT Publication No. WO 94/18318.

Upon identification of a compound that modulates the interaction of a cell surface receptor of interest, such a compound can be further investigated to test for an ability to alter the allergenic or inflammatory response. In particular, for example, the compounds identified via the present methods can be further tested in vivo in accepted animal models.

Computer modeling and searching technologies permit identification of compounds, or the improvement of already identified compounds, that can modulate the interaction receptor interactions. Having identified such a compound or composition, the binding sites or regions are identified. The binding site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the binding site by finding where on the target the complexed ligand is found. Next, the three dimensional geometric structure of the binding site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the binding site structure as determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the binding site, either experimentally, by modeling, or by a combination, candidate modulating compounds can be identified by searching databases containing compounds along with information on their molecular structure. Such a search seeks compounds having structures that match the determined binding site structure and that interact with the groups defining the active site. Such a search can be manual, but is preferably computer assisted. These compounds found from this search are potential useful compounds.

Alternatively, these methods can be used to identify improved pharmaceutically active compounds from an already known modulating compound or ligand. The composition of the known compound can be modified and the structural effects of modification can be determined using the experimental and computer modeling methods described above applied to the new composition. The altered structure is then compared to the binding site structure of the previously known compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the binding sites will be apparent to those of skill in the art. Examples of molecular modeling systems are the CHARMm and QUANTA programs (Polygen Corporation, Waltham, Mass.). CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modeling of drugs interactive with specific proteins, such as Rotivinen et al.,) 1988, Acta Pharmaceutical Fennica 97:159-166); Ripka (1988 New Scientist 54-57); McKinaly and Rossmann (1989, Annu. Rev. Pharmacol. Toxiciol. 29:111-122); Perry and Davies, OSAR: Quantitative Structure-Activity Relationships in Drug Design pp. 189-193 Alan R. Liss, Inc. 1989; Lewis and Dean (1989, Proc. R. Soc. Lond. 236:125-140 and 141-162); and, with respect to a model receptor for nucleic acid components, Askew, et al., (1989, J. Am. Chem. Soc. 111:1082-1090). Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can also be adapted to design of drugs specific to regions of DNA or RNA, once that region is identified.

Antibodies that specifically recognize one or more epitopes of the targeted receptor are also useful according to the practice of the invention. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

Compounds Useful for Suppressing, Inhibiting or Preventing Amyloid Formation Enhancers of Intracellular cAMP

Compounds described below are useful for suppressing, inhibiting or preventing amyloid formation. Some of these compounds promote, or increase the level of intracellular cyclic adenosine monophosphate (cAMP) in a cell. Thus, another embodiment of the present invention is a method of suppressing, inhibiting or preventing cell-mediated amyloid formation by contacting a cell with a compound that promotes or increases the level of intracellular cAMP in the cell. In a preferred embodiment, the present invention provides a method of suppressing, inhibiting or preventing amyloid formation mediated by microglia, comprising contacting the microglia with an effective amount of an enhancer of intracellular cAMP. Yet another preferred embodiment is a method of suppressing, inhibiting or preventing amyloid formation mediated by a cell of macrophage lineage, comprising contacting the cell with an effective amount of at least one enhancer of intracellular cAMP. In a typical case, these methods are successfully practiced to prevent or treat Alzheimer's disease.

An enhancer of intracellular cAMP shall generally refer to a compound that increases the level of intracellular cAMP above the level present in the absence of the enhancer. Examples of an enhancer of intracellular cAMP include, but are not limited to, forskolin, rolipram, 8-bromo-cAMP, dibutyryl-cAMP and other analogs or derivatives thereof. Thus, another embodiment of the present invention is a method of preventing cell-mediated amyloid formation by contacting the cell with an effective amount of at least one compound selected from the group consisting of forskolin, rolipram, 8-bromo-cAMP, dibutyryl-cAMP, cAMP and one of the analogs or derivatives thereof. Yet another embodiment of the present invention is a method of suppressing, inhibiting or preventing activity of a microglial cell or a cell of macrophage lineage, comprising contacting the microglial cell or the cell of macrophage lineage with a compound selected from the group consisting of forskolin, rolipram, 8-bromo-cAMP, cAMP, dibutyryl-cAMP, cAMP and one of the analogs or derivatives thereof.

An enhancer of intracellular cAMP may also increase the level of intracellular cAMP by preventing metabolism of intracellular cAMP. Examples of such enhancers of intracellular cAMP include, but are not limited to, an inhibitor of a cAMP phosphodiesterase. Thus, one embodiment of the present invention is a method of suppressing, preventing or inhibiting amyloid formation mediated by a cell, comprising contacting the cell with an effective amount of an inhibitor of a cAMP phosphodiesterase. A preferred embodiment of the present invention is a method of suppressing, preventing or inhibiting amyloid formation mediated by a microglial cell or a cell of macrophage lineage, comprising contacting the microglial cell or the cell of macrophage lineage, with an effective amount of an inhibitor of a cAMP phosphodiesterase.

A number of phosphodiesterases (also known as PDEs) have been characterized. The activities of PDE type 1 (including subtypes a, b, and c), type 2a, type 3 (including subtypes a, and b), type 4 (including subtypes a, b, c, and d), type 7 (including subtypes a, and b, type 8 (including subtypes a, and b), type 10, and type 11 have been shown to decrease the level of intracellular cAMP

Accordingly, one embodiment of the present invention is an inhibitor of a cAMP phosphodiesterase selected from the group consisting of PDE1, PDE2, PDE3, PDE4, PDE7, PDE8, PDE10, PDE11, their respective subtypes, and one of the derivatives or analogs thereof. In a more preferred embodiment, an inhibitor of a cAMP phosphodiesterase is an inhibitor of PDE4, PDE7 or PDE10. In an even more preferred embodiment, an inhibitor of cAMP phosphodiesterase is an inhibitor of PDE4a, PDE4b, PDE4c or PDE4d.

An inhibitor of PDE can be obtained by a variety of methods commonly known to a person skilled in the art. Inhibitors of PDE have been described previously. For example, U.S. Pat. Nos. 6,649,640, 6,649,633 and 6,649,631 provide examples of inhibitors of PDE4. Further examples of inhibitors of PDE4 can be found in U.S. Patent Application Publication Nos. 20030104974. Examples of inhibitors of PDE10 include papaverine and further examples can be found in U.S. Pat. No. 6,538,029, U.S. Patent Application Publication Nos. 20030008806, 20030018047, and 20030032579. Examples of inhibitors of PDE7 can be found in U.S. Patent Application Publication No. 2002198198 and International Patent Application Publication Nos. WO02/076953, WO 02/074754. Furthermore, a person skilled in the art may be able to identify additional compounds using the method described in U.S. Pat. Nos. 6,350,603, 6,635,436, and 6,368,815.

Thus, one embodiment of the present invention is a method of suppressing, inhibiting or preventing amyloid formation mediated by a microglial cell or a cell of macrophage lineage in a subject, preferably in a mammal, comprising administering to such subject an effective amount of an inhibitor of PDE. One preferred inhibitor of PDE is an inhibitor of PDE 4, such as rolipram, and another preferred inhibitor of PDE is an inhibitor of PDE10.

Agonists of Prostaglandin E2 Receptors

Another aspect of the present invention relates to the use of agonists of a prostaglandin E2 (PGE2) receptor. The prostaglandin E2 receptor has several subtypes, including, but not limited to, subtypes 1, 2, 3 and 4 (also known as EP1, EP2, EP3, and EP4 respectively). According to the present invention, compounds that are agonists of prostaglandin E2 receptors are useful for suppressing, inhibiting or preventing cell-mediated amyloid formation. In particular, according to the present invention, agonists of subtypes EP2 and EP4 are especially useful for suppressing, inhibiting or preventing cell-mediated amyloid formation.

Examples of an agonist of the prostaglandin E2 receptor subtype EP1 can be found in U.S. Pat. No. 6,448,290 and Patent Application Publication Nos. WO 96/06822, WO 96/11902 and EP 752421-A1. Additional compounds may be identified in accordance with methods described in U.S. Pat. No. 6,440,680. Preparing, synthesizing, using and/or administering the agonists of the prostaglandin E2 receptors are further described in U.S. Pat. Nos. 6,610,719, 6,642,266, 6,649,657, 6,492,412, 6,426,359 and 6,288,120, U.S. Patent Application Publication No. US 2003/0216445, PCT Application, Publication Nos. WO 03/064391, WO 03/045351 and WO 99/19300. Each of these references is hereby incorporated by reference in its entirety.

Examples of an agonist of the prostaglandin EP4 receptor can be found in U.S. Pat. Nos. 6,642,266, 6,610,719, 6,552,067, all of which are hereby incorporated in their entireties by reference.

Examples of an agonist of the prostaglandin EP2 receptor can be found in U.S. Pat. Nos. 6,649,657, 6,426,359, 6,492,412, all of which are hereby incorporated in their entireties by reference.

Accordingly, detailed examples of compounds useful according to the practice of the invention are provided, in particular, examples of agonists of the prostaglandin E2 receptor which include, without limitation, compounds which may be represented, for example, by formula I

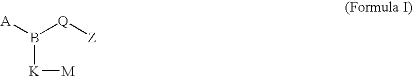

or a pharmaceutically-acceptable salt or prodrug thereof wherein either (i):

B is N;

A is (C₁-C₆)alkylsulfonyl, (C₃-C₇)cycloalkylsulfonyl, (C₃-C₇)cycloalkyl(C₁-C₆)alkylsulfonyl, said A moieties optionally mono-, di- or tri-substituted on carbon independently with hydroxy, (C₁-C₄)alkyl or halo;

Q is

—(C₂-C₆)alkaline-W—(C₁-C₃)alkaline-,

—(C₃-C₈)alkaline-, said —(C₃-C₈)alkaline-optionally substituted with up to four substituents independently selected from fluoro or (C₁-C₄)alkyl,

—X—(C₁-C₅)alkaline-,

—(C₁-C₅)alkaline-X—,

—(C₁-C₃)alkaline-X—(C₁-C₃)alkaline-,

—(C₂-C₄)alkaline-W—X—(C₀-C₃)alkaline-,

—(C₂-C₄)alkaline-X—W—(C₁-C₃)alkaline-,

—(C₂-C₅)alkaline-W—X—W—(C₁-C₃)alkaline-, wherein the two occurrences of W are independent of each other,

—(C₁-C₄)alkylene-ethenylene-(C₁-C₄)alkaline-,

—(C₁-C₄)alkylene-ethenylene-(C₀-C₂)alkaline-X—(C₀-C₅)alkaline-,

—(C₁-C₄)alkylene-ethenylene-(C₀-C₂)alkaline-X—W—(C₁-C₃)alkaline-,

—(C₁-C₄)alkylene-ethynylene-(C₁-C₄)alkaline-, or

—(C₁-C₄)alkylene-ethynylene-X—(C₀-C₃)alkaline-;

W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;

X is a five or six membered aromatic ring optionally having one or two heteroatoms selected independently from oxygen, nitrogen, and sulfur; said ring optionally mono-, or di-substituted independently with halo, (C₁-C₃)alkyl, trifluoromethyl, trifluoromethyloxy, difluoromethyloxy, hydroxyl, (C₁-C₄)alkoxy, or carbamoyl;

Z is carboxyl, (C₁-C₆)alkoxycarbonyl, tetrazolyl, 1,2,4-oxadiazolyl, 5-oxo-1,2,4-oxadiazolyl, (C₁-C₄)alkylsulfonylcarbamoyl or phenylsulfonylcarbamoyl;

K is a bond, (C₁-C₈)alkaline, thio(C₁-C₄)alkaline or oxy(C₁-C₄)alkaline, said (C₁-C₈)alkaline optionally mono-unsaturated and wherein K is optionally mono-, di- or tri-substituted independently with fluoro, methyl or chloro;

M is —Ar, —Ar¹—V—Ar², —Ar¹—S—Ar² or —Ar¹—O—Ar² wherein Ar, Ar¹ and Ar² are each independently a partially saturated, fully saturated or fully unsaturated five to eight membered ring optionally having one to four heteroatoms selected independently from oxygen, sulfur and nitrogen, or, a bicyclic ring consisting of two fused partially saturated, fully saturated or fully unsaturated five or six membered rings, taken independently, optionally having one to four heteroatoms selected independently from nitrogen, sulfur and oxygen;

said Ar, Ar¹ and Ar² moieties optionally substituted, on one ring if the moiety is monocyclic, or one or both rings if the moiety is bicyclic, on carbon with up to three substituents independently selected from R¹, R² and R³ wherein R¹, R² and R³ are hydroxy, nitro, halo, (C₁-C₆)alkoxy, (C₁-C₄)alkoxy(C₁-C₄)alkyl, (C₁-C₄)alkoxycarbonyl, (C₁-C₇)alkyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;

R¹, R² and R³ are optionally mono-, di- or tri-substituted on carbon independently with halo or hydroxy; and

V is a bond or (C₁-C₃)alkaline optionally mono- or di-substituted independently with hydroxy or fluoro

with the proviso that when K is (C₂-C₄)alkaline and M is Ar and Ar is cyclopent-1-yl, cyclohex-1-yl, cyclohept-1-yl or cyclooct-1-yl then said (C₅-C₈)cycloalkyl substituents are not substituted at the one position with hydroxy;

or (ii):

B is N;

A is (C₁-C₆)alkanoyl, or (C₃-C₇)cycloalkyl(C₁-C₆)alkanoyl, said A moieties optionally mono-, di- or tri-substituted independently on carbon with hydroxy or halo;

### Q is

—(C₂-C₆)alkaline-W—(C₁-C₃)alkaline-,

—(C₄-C₈)alkaline-, said —(C₄-C₈)alkaline- optionally substituted with up to four substituents independently selected from fluoro or (C₁-C₄)alkyl,

—X—(C₂-C₅)alkaline-,

—(C₁-C₅)alkaline-X—,

—(C₁-C₃)alkaline-X—(C₁-C₃)alkaline-,

—(C₂-C₄)alkaline-W—X—(C₀-C₃)alkaline-,

—(C₀-C₄)alkaline-X—W—(C₁-C₃)alkaline-,

—(C₂-C₅)alkaline-W—X—W—(C₁-C₃)alkaline-, wherein the two occurrences of W are independent of each other,

—(C₁-C₄)alkylene-ethenylene-(C₁-C₄)alkaline-,

—(C₁-C₄)alkylene-ethenylene-(C₀-C₂)alkaline-X—(C₀-C₅)alkaline-,

—(C₁-C₄)alkylene-ethenylene-(C₀-C₂)alkaline-X—W—(C₁-C₃)alkaline-,

—(C₁-C₄)alkylene-ethynylene-(C₁-C₄)alkaline-, or

—(C₁-C₄)alkylene-ethynylene-X—(C₀-C₃)alkaline-;

W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;

X is a five or six membered aromatic ring optionally having one or two heteroatoms independently selected from oxygen, nitrogen, and sulfur; said ring optionally mono-, or di-substituted independently with halo, (C₁-C₃)alkyl, trifluoromethyl, trifluoromethyloxy, difluoromethyloxy, hydroxyl, (C₁-C₄)alkoxy, or carbamoyl;

Z is carboxyl, (C₁-C₆)alkoxycarbonyl, tetrazolyl, 1,2,4-oxadiazolyl, 5-oxo-1,2,4-oxadiazolyl, (C₁-C₄)alkylsulfonylcarbamoyl or phenylsulfonylcarbamoyl;

K is (C₁-C₈)alkaline, thio(C₁-C₄)alkaline or oxy(C₁-C₄)alkaline, said (C₁-C₈)alkaline optionally mono-unsaturated and wherein K is optionally mono-, di- or tri-substituted independently with fluoro, methyl or chloro;

M is —Ar, —Ar¹—V—Ar, —Ar¹—S—Ar² or —Ar¹—O—Ar² wherein Ar, Ar¹ and Ar² are each independently a partially saturated, fully saturated or fully unsaturated five to eight membered ring optionally having one to four heteroatoms selected independently from oxygen, sulfur and nitrogen, or, a bicyclic ring consisting of two fused partially saturated, fully saturated or fully unsaturated five or six membered rings, taken independently, optionally having one to four heteroatoms selected independently from nitrogen, sulfur and oxygen;

said Ar, Ar¹ and Ar² moieties optionally substituted, on one ring if the moiety is monocyclic, or one or both rings if the moiety is bicyclic, on carbon with up to three substituents independently selected from R¹, R² and R³ wherein R¹, R² and R³ are H, hydroxy, nitro, halo, (C₁-C₆)alkoxy, (C₁-C₄)alkoxy(C₁-C₄)alkyl, (C₁-C₄)alkoxycarbonyl, (C₁-C₇)alkyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;

R¹, R² and R³ are optionally mono-, di- or tri-substituted on carbon independently with halo or hydroxy; and

V is a bond or (C₁-C₃)alkaline optionally mono- or di-substituted independently with hydroxy or fluoro

with the proviso that when K is (C₂-C₄)alkaline and M is Ar and Ar is cyclopent-1-yl, cyclohex-1-yl, cyclohept-1-yl or cycloct-1-yl then said (C₅-C₈)cycloalkyl substituents are not substituted at the one position with hydroxy

and with the proviso that 6-[(3-phenyl-propyl)-(2-propyl-pentanoyl)-amino]-hexanoic acid and its ethyl ester are not included

or (iii):

B is C(H);

A is (C₁-C₆)alkanoyl, or (C₃-C₇)cycloalkyl(C₁-C₆)alkanoyl, said A moieties optionally mono-, di- or tri-substituted on carbon independently with hydroxy or halo;

Q is

—(C₂-C₆)alkaline-W—(C₁-C₃)alkaline-,

—(C₄-C₈)alkaline-, said —(C₄-C₈)alkaline- optionally substituted with up to four substituents independently selected from fluoro or (C₁-C₄)alkyl,

—X—(C₁-C₅)alkaline-,

—(C₁-C₅)alkaline-X—,

—(C₁-C₃)alkaline-X—(C₁-C₃)alkaline-,

—(C₂-C₄)alkaline-W—X—(C₀-C₃)alkaline-,

—(C₀-C₄)alkaline-X—W—(C₁-C₃)alkaline-,

—(C₂-C₅)alkaline-W—X—W—(C₁-C₃)alkaline-, wherein the two occurrences of W are independent of each other,

—(C₁-C₄)alkylene-ethenylene-(C₁-C₄)alkaline-,

—(C₁-C₄)alkylene-ethenylene-(C₀-C₂)alkaline-X—(C₀-C₅)alkaline-,

—(C₁-C₄)alkylene-ethenylene-(C₀-C₂)alkaline-X—W—(C₁-C₃)alkaline-,

—(C₁-C₄)alkylene-ethynylene-(C₈-C₄)alkaline-, or

—(C₁-C₄)alkylene-ethynylene-X—(C₀-C₃)alkaline-;

W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;

X is a five or six membered aromatic ring optionally having one or two heteroatoms selected independently from oxygen, nitrogen and sulfur, said ring optionally mono-, or di-substituted independently with halo, (C₁-C₃)alkyl, trifluoromethyl, trifluoromethyloxy, difluoromethyloxy, hydroxyl, (C₁-C₄)alkoxy, or carbamoyl;

Z is carboxyl, (C₁-C₆)alkoxycarbonyl, tetrazolyl, 1,2,4-oxadiazolyl, 5-oxo-1,2,4-oxadiazolyl, (C₁-C₄)alkylsulfonylcarbamoyl or phenylsulfonylcarbamoyl;

K is a bond, (C₁-C₈)alkaline, thio(C₁-C₄)alkaline, (C₄-C₇)cycloalkyl(C₁-C₆)alkaline or oxy(C₁-C₄)alkaline, said (C₁-C₈)alkaline optionally mono-unsaturated and wherein K is optionally mono-, di- or tri-substituted independently with fluoro, methyl or chloro;

M is —Ar, —Ar¹—V—Ar², —Ar¹—S—Ar² or —Ar¹—O—Ar² wherein Ar, Ar¹ and Ar² are each independently a partially saturated, fully saturated or fully unsaturated five to eight membered ring optionally having one to four heteroatoms selected independently from oxygen, sulfur and nitrogen, or, a bicyclic ring consisting of two fused partially saturated, fully saturated or fully unsaturated five or six membered rings, taken independently, optionally having one to four heteroatoms selected independently from nitrogen, sulfur and oxygen;

said Ar, Ar¹ and Ar² moieties optionally substituted, on one ring if the moiety is monocyclic, or one or both rings if the moiety is bicyclic, on carbon with up to three substituents independently selected from R¹, R² and R³ wherein R¹, R² and R³ are H, hydroxy, nitro, halo, (C₁-C₆)alkoxy, (C₁-C₄)alkoxy(C₁-C₄)alkyl, (C₁-C₄)alkoxycarbonyl, (C₁-C₇)alkyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;

R¹, R² and R³ are optionally mono-, di- or tri-substituted independently on carbon with halo or hydroxy; and

V is a bond or (C₁-C₃)alkaline optionally mono- or di-substituted independently with hydroxy or fluoro

with the proviso that when K is (C₂-C₄)alkaline and M is Ar and Ar is cyclopent-1-yl, cyclohex-1-yl, cyclohept-1-yl or cyclooct-1-yl then said (C₅-C₈)cycloalkyl substituents are not substituted at the one position with hydroxy.

Preferred compounds include the following

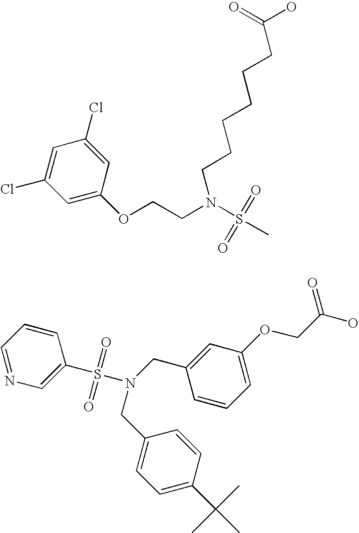

(3-{[(4-tert-Butyl-benzyl)-(pyridine-3-sulfonyl)-amino]-methyl}-phenoxy)-acetic acid, and

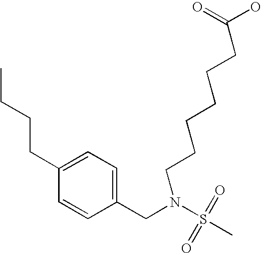

Compounds that are Effective Outside the Described Camp-Mediated Mechanisms

(5) As aforementioned, compounds that interact positively with (i.e. are agonists for) nuclear receptors in brain microglial cells, including glucocorticoids (for example, dexamethasone, prednisolone), non-glucocorticoid compounds that act as agonists at the glucocorticoid receptor, and dissociated agonists of glucocorticoid receptors (“DAGRs”, see U.S. Pat. No. 6,506,766). Certain compounds useful for suppressing, inhibiting, or preventing amyloid formation include, for example, corticosterone and other glucocorticoids that bind to and fully activate (i.e. are agonists for) the glucocorticoid receptor. Other useful compounds are glucocorticoid analogs or mimics that cause ligand-dependent alterations in glucocorticoid receptor conformations, resulting in retention of the ability to suppress, inhibit or prevent amyloid formation, while minimizing, for example, bone and diabetic side effects.

An additional class of compounds useful in all aspects of the invention is represented by the HMG CoA reductase inhibitors (statins), and which show very positively in the MFA assay, representative examples being atorvastatin, mevastatin, and lovastatin.

Pharmaceutical Compositions and their Use

Another aspect of the present invention is methods of preparing or administering a pharmaceutical composition useful for treating or preventing a disease or condition caused by or exhibiting cell-mediated amyloid formation, or a disease or condition caused by or relating to the activities of microglia or cells of macrophage lineage.

The pharmaceutical compositions of the present invention comprise any one or more of the above-described compounds, or a pharmaceutically acceptable salt thereof, together with a pharmaceutically acceptable carrier in accordance with the properties and expected performance of such a carrier, as is well-known in the art.

The term “carrier” as used herein includes acceptable diluents, excipients, adjuvants, vehicles, solubilization aids, viscosity modifiers, preservatives, and other agents well known to the artisan for providing favorable properties in the final pharmaceutical composition.

The dosage and dose rate of the compounds identified in the present invention effective for treating or preventing a disease or condition exhibiting, caused by or relating to amyloid formation, or a disease or condition caused by, exhibiting or relating to the activities of microglia or cells of macrophage lineage, will depend on a variety of factors, such as the nature of the inhibitor, the size of the patient, the goal of the treatment, the nature of the pathology to be treated, the specific pharmaceutical composition used, and the observations and conclusions of the treating physician.

For example, where the dosage form is oral, e.g., a tablet or capsule, suitable dosage levels may be between about 0.1 μg/kg and about 50.0 mg/kg body weight per day, preferably between about 1.0 μg/kg and about 5.0 mg/kg body weight per day, more preferably between about 10.0 μg/kg and about 1.0 mg/kg of body weight per day, and most preferably between about 20.0 μg/kg and about 0.5 mg/kg of body weight per day of the active ingredient.

Using representative body weights of 10 kg and 100 kg in order to illustrate the range of daily aerosolized topical dosages that might be used as described above, suitable dosage levels of a compound identified in the present invention will be between about 1.0-10.0 μg and 500.0-5000.0 mg per day, preferably between about 5.0-50.0 μg and 5.0-50.0 mg per day, more preferably between about 100.0-1000.0 μg and 10.0-100.0 mg per day, and most preferably between about 200.0-2000.0 μg and about 5.0-50.0 mg per day of the active ingredient. These ranges of dosage amounts represent total dosage amounts of the active ingredient per day for a given patient. The number of times per day that a dose is administered will depend upon such pharmacological and pharmacokinetic factors as the half-life of the active ingredient, which reflects its rate of catabolism and clearance, as well as the minimal and optimal blood plasma or other body fluid levels of said active ingredient attained in the patient that are required for therapeutic efficacy.

Numerous other factors must also be considered in deciding upon the number of doses per day and the amount of active ingredient per dose that will be administered. Not the least important of such other factors is the individual response of the patient being treated. Thus, for example, where the active ingredient is administered topically via aerosol inhalation into the lungs, from one to four doses consisting of acuations of a dispensing device, i.e., “puffs” of an inhaler, will be administered each day, each dose containing from about 50.0 μg to about 10.0 μg of active ingredient.

Additional detailed information is as follows.

The Drug Substance

Pharmaceutically acceptable salts of the compounds of formula I include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002). Pharmaceutically acceptable salts of compounds of formula I, for example, may be prepared by one or more of three methods:

(i) by reacting the compound of formula I with the desired acid or base;

(ii) by removing an acid- or base-labile protecting group from a suitable precursor of the compound of formula I or by ring-opening a suitable cyclic precursor, for example, a lactone or lactam, using the desired acid or base; or

(iii) by converting one salt of the compound of formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water. A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula I include references to salts, solvates, multi-component complexes and liquid crystals thereof and to solvates, multi-component complexes and liquid crystals of salts thereof. The compounds of the invention include compounds of formula I as hereinbefore defined, including all polymorphs and crystal habits thereof, prodrugs and isomers thereof (including optical, geometric and tautomeric isomers) as hereinafter defined and isotopically-labeled compounds of formula I.

As indicated, so-called ‘prodrugs’ of the compounds of formula I are also within the scope of the invention. Thus certain derivatives of compounds of formula I which may have little or no pharmacological activity themselves can, when administered into or onto the body, be converted into compounds of formula I having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula I with certain moieties known to those skilled in the art as ‘pro-moieties’ as described, for example, in Design of Prodrugs by H. Bundgaard (Elsevier, 1985). Some examples of prodrugs in accordance with the invention include

(i) where the compound of formula I contains a carboxylic acid functionality

(—COOH), an ester thereof, for example, a compound wherein the hydrogen of the carboxylic acid functionality of the compound of formula (I) is replaced by (C₁-C₈)alkyl;

(ii) where the compound of formula I contains an alcohol functionality (—OH), an ether thereof, for example, a compound wherein the hydrogen of the alcohol functionality of the compound of formula I is replaced by (C₁-C₆)alkanoyloxymethyl; and

(iii) where the compound of formula I contains a primary or secondary amino functionality (—NH₂ or —NHR where R≠H), an amide thereof, for example, a compound wherein, as the case may be, one or both hydrogens of the amino functionality of the compound of formula I is/are replaced by (C₁-C₁₀)alkanoyl.

Further examples of replacement groups in accordance with the foregoing examples and examples of other prodrug types may be found in the aforementioned references. Moreover, certain compounds of formula I may themselves act as prodrugs of other compounds of formula I.

Also included within the scope of the invention are metabolites of compounds of formula I, that is, compounds formed in vivo upon administration of the drug. Some examples of metabolites in accordance with the invention include

(i) where the compound of formula I contains a methyl group, an hydroxymethyl derivative thereof (—CH₃->—CH₂OH):

(ii) where the compound of formula I contains an alkoxy group, an hydroxy derivative thereof (—OR->—OH);

(iii) where the compound of formula I contains a tertiary amino group, a secondary amino derivative thereof (—NR¹R²->—NHR¹ or —NHR²);

(iv) where the compound of formula I contains a secondary amino group, a primary derivative thereof (—NHR¹->—NH₂);

(v) where the compound of formula I contains a phenyl moiety, a phenol derivative thereof (-Ph->-PhOH); and

(vi) where the compound of formula I contains an amide group, a carboxylic acid derivative thereof (—CONH₂->COOH).

Compounds of formula I containing one or more asymmetric carbon atoms can exist as two or more stereoisomers. Where a compound of formula I contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula I, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of formula I wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula I, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula I can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Also within the scope of the invention are intermediate compounds of formula II as hereinbefore defined, all salts, solvates and complexes thereof and all solvates and complexes of salts thereof as defined hereinbefore for compounds of formula I. The invention includes all polymorphs of the aforementioned species and crystal habits thereof.

When preparing compounds of formula I in accordance with the invention, it is open to a person skilled in the art to routinely select the form of compound of formula II which provides the best combination of features for this purpose. Such features include the melting point, solubility, processability and yield of the intermediate form and the resulting ease with which the product may be purified on isolation.

The Drug Product

The compounds of formula I should be assessed for their biopharmaceutical properties, such as solubility and solution stability (across pH), permeability, etc., in order to select the most appropriate dosage form and route of administration for treatment of the proposed indication. Compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

They may be administered alone or in combination with one or more other compounds of the invention or in combination with one or more other drugs (or as any combination thereof). Generally, they lbut will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Oral Administration

The compounds of the invention may be administered orally. Oral administration may involve swallowing, so that the compound enters the gastrointestinal tract, and/or buccal, lingual, or sublingual administration by which the compound enters the blood stream directly from the mouth. Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches. Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet. The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form. Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet. Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

The compound of formula I may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula I may be in the form of multiparticulate beads. The film-forming polymer may be selected from natural polysaccharides, proteins, or synthetic hydrocolloids and is typically present in the range 0.01 to 99 weight %, more typically in the range 30 to 80 weight %. Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified controlled release. Controlled release formulations include Modified release formulations include delayed-, sustained-, pulsed-, controlled-, or tragettedtargeted and programmed release. Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et a/(2001). The use of chewing gum to achieve controlled release is described in WO 00/35298.

Drug Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

An example of a needle free injection is a powderjet to provide an example of suitable technologies) formulations are typically aqueous solutions which may contain excipients such as salts, carbohydrates and buffering agents (preferably. to a pH of from 3 to 9), but, for some applications, they may be more suitably formulated as a sterile non-aqueous solution or as powdered a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art. The solubility of compounds of formula I used in the preparation of parenteral solutions may be increased by the use of appropriate formulation techniques, such as the incorporation of solubility-enhancing agents. Formulations for use with needle-free injection administration comprise a compound of the invention in powdered form in conjunction with a suitable vehicle such as sterile, pyogen-free water.

Formulations for parenteral administration may be formulated to be immediate and/or modified controlled release. Controlled release formulations include Modified release formulations include delayed-, sustained-, pulsed-, controlled-, or tragettedtargeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of the invention may also be administered topically, (intra)dermally, or transdermally to the skin or mucosa. Typical formulations for this purpose tio include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection. Topical administration may also be achieved using a patch, such as a transdermal iontophoretic patch. Formulations for topical administration may be formulated to be immediate and/or modified controlled release. Controlled release formulations include Modified release formulations include delayed-, sustained-, pulsed-, controlled-, or tragettedtargeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin. The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified controlled release using, for example, PGLA. Controlled release formulations include Modified release formulations include delayed-, sustained-, pulsed-, controlled-, or tragetted and programmed release.

In the case of dry powder inhalers and aerosols, the dosage unit is determined by means of a valve which delivers a metered amount. Units in accordance with the invention are typically arranged to administer a metered dose or “puff” containing the compound of formula I. The overall daily dose will typically be in the range 50 μg to 2000 mg which may be administered in a single dose or, more usually, as divided doses throughout the day.

The compounds of the invention may also be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus the kit of the invention comprises two or more separate pharmaceutical compositions, at least one of which contains a compound of formula I in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 0.001 mg to 2000 mg depending, of course, on the mode of administration. These dosages are based on an average human subject having a weight of about 60 kg to 70 kg. The physician will readily be able to determine doses for subjects whose weight falls outside this range, such as infants and the elderly.

In regard of the present specification, all patents and publications cited herein are incorporated by reference, as if fully set forth.

1. A method of identifying a compound useful for suppressing amyloid formation mediated by a cell, comprising contacting the cell with a candidate compound, adding amyloidogenic peptides, and comparing the level of amyloid formation mediated by the cell in the presence and absence of the candidate compound.

2. The method of claim 1, wherein the cell is a microglial cell.

3. The method of claim 1, wherein the cell is of macrophage lineage.

4. The method of claim 1, wherein the cell is derived from a mammalian brain.

5. The method of claim 4, wherein the cell is a microglial cell derived from a mammalian brain.

6. The method of claim 1, wherein the amyloidogenic peptides are derived from amyloid precursor protein.

7. The method of claim 1, wherein the amyloidogenic peptides are selected from the group consisting of Aβ (1-43) peptide, Aβ (1-42) peptide, Aβ (1-40) peptide and Aβ (1-17) peptide.

8. The method of claim 1, wherein a smooth muscle cell is also present and wherein the amyloidogenic peptides are extruded from the smooth muscle cell.

9. The method of claim 8, wherein the smooth muscle cell is a vascular smooth muscle cell.

10. A method of identifying a compound that suppresses or inhibits cell-mediated amyloid formation, comprising
(a) preparing at least one cell capable of mediating amyloid formation from amyloidogenic peptides,
(b) mixing the cell with an effective amount of a candidate compound and amyloidogenic peptides,
(c) incubating the mixture for a sufficient length of time to allow amyloid formation,
(d) measuring the level of amyloid formation, and
(e) comparing the level of amyloid formation to the level in the absence of a candidate compound.

11. The method as described in claim 10, wherein the cell is a microglial cell or a cell of macrophage lineage.

12. The method as described in claim 10, wherein the cell is derived from a mammalian brain or nervous system.

13. The method as described in claim 10, wherein the cell is a microglial cell derived from a mammalian brain.

14. The method as described in claim 10, wherein the method is carried out in presence of smooth muscle cells and wherein the amyloidogenic peptides are provided by the smooth muscle cells.

15. The method as described in claim 10, wherein the amyloidogenic peptides are selected from the group consisting of Aβ (1-43) peptide, AD (1-42) peptide, Aβ (1-40) peptide, and Aβ (1-17) peptide.

16. The method as described in claim 15, wherein the amyloidogenic peptides are prions.

17. The method as described in claim 10, wherein a labeling agent is used to measure the level of amyloid formation.

18. The method as described in claim 17, wherein the labeling agent is thioflavin S or Congo red.

19. The method as described in claim 17, wherein the labeling agent is an antibody that recognizes amyloid aggregates.

20. A method of suppressing or inhibiting cell-mediated amyloid formation in a subject in need thereof, comprising administering to the subject an effective amount of at least one compound selected from the group consisting of an enhancer of intracellular cAMP, an inhibitor of cAMP-specific phosphodiesterase, and an agonist of prostaglandin E2 receptors.

21. A method of suppressing or inhibiting cell-mediated amyloid formation in a subject in need thereof, comprising administering to the subject an effective amount of at least one enhancer of intracellular cAMP.
